(19) United States
(12) Patent Application Publication
(10) Pub. No.: US 2003/0224375 A1
(43) Pub. Date: Dec. 4, 2003

(54) Assays for evaluating the function of rna helicases

(76) Inventors: Anna Marie Pyle, New York, NY (US); Eckhard Jankowsky, New York, NY (US)

Correspondence Address: John P White, Cooper & Dunham, 1185 Avenue of the Americas, New York, NY, 10036, US

(21) Appl. No.: 10/182,362

(22) PCT Filed: Jan. 26, 2001
(86) PCT No.: PCT/US01/02734

(51) Int. Cl. C12Q 1/68

(52) U.S. Cl. 435/6

(57) ABSTRACT

The present invention provides a method for detecting the release of a single-stranded RNA from an RNA duplex which comprises admixing an RNA helicase with the RNA duplex under conditions permitting the RNA helicase to unwind the RNA duplex and release single-stranded RNA. wherein the RNA duplex comprises a first RNA having a first label attached thereto and a second RNA, such first label being capable of producing a luminescent energy pattern when the first RNA is present in the RNA duplex which differs from the luminescent energy pattern produced when the first RNA is not present in the RNA duplex, subjecting the admixture to conditions which permit (i) the RNA helicase to unwind the RNA duplex and release single-stranded RNA, and (ii) the first label to produce luminescent energy. and detecting a change in the luminescent energy pattern produced by the first label so as to thereby detect release of single-stranded RNA from the RNA duplex.

This application is a continuation-in-part of U.S. Ser. No. 09/492,954, filed Jan. 27, 2000, the contents of which are hereby incorporated by reference.

The invention disclosed herein was made with Government support under NIH Grant No. R0150313 from the Department of Health and Human Services. Accordingly, the U.S. Government has certain. rights in this invention.

### BACKGROUND ON THE INVENTION

Throughout this application, various publications are referenced by author and date. Full citations for these publications may be found listed alphabetically at the end of the specification immediately preceding Sequence Listing and the claims. The disclosures of these publications in their entireties are hereby incorporated by reference into this application in order to more fully describe the state of the art as known to those skilled therein as of the date of the invention described and claimed herein.

RNA conformation is of elemental importance in RNA-induced catalysis, as well as RNA interactions with other cellular components. Many central components of gene expression and RNA metabolism occur in large ribonucleoprotein complexes, most notably the ribosome and spliceosome. Within these large complexes, alternative conformations of specific RNAs have been demonstrated and the alteration of RNA conformation is believed to play a critical role in enabling driving and assuring the fidelity of the catalytic reactions these complexes perform (Staley and Guthrie, 1998). Therefore, understanding the factors capable of inducing RNA conformational alterations within these complexes is likely to be central to an overall appreciation of mechanisms by which they execute their intricate and demanding functions.

By exploring the effect of the duplex length of the substrates on the unwinding reaction under defined reaction conditions, the response of unwinding rate and amplitude to the duplex length can provide information about processivity and directionality (FIG. 5). Thus, the requirements for an experimental setup to probe and quantify processivity and directionality are to develop a substrate system where only the duplex length is variable and, using this substrate system, establish experimental conditions where rate and/or amplitude provide information about processivity and directionality.

RNA helicases of the DExH/D family play an essential role in viral replication and cellular RNA metabolism, including central functions in RNA splicing, translation and regulation of gene expression. Despite the importance of these proteins, their RNA helicase activity has not been subjected to enzymological study. Basic knowledge of cellular metabolism is therefore constrained by a limited understanding of reaction mechanism by motor proteins in the RNA helicase family. To address this problem, mechanistic studies have been initiated on two viral DExH/D proteins which are part of the RNA helicase family: NPH-II from Vaccinia and NS3-4A from Hepatitis C Virus (HCV). The NPH-II protein is shown to be a processive, directional RNA helicase with specific roles for both the binding and hydrolysis of ATP.

Having established qualitative features of NPH-II activity, the use of direct and stopped-flow kinetic methods to determine the quantitative kinetic parameters such as translocation rates, reaction step size, processivity, helicase binding, ATP binding and hydrolic rate constants that describe the framework for catalytic activity of this prototypical RNA helicase. All aspects of cellular RNA metabolism and processing involve DExH/D proteins, which are a family of enzymes that unwind or manipulate RNA in an ATP-dependent fashion (de la Cruz, et al., 1999) . DExH/D proteins are also essential for the replication of many viruses, and therefore provide targets for the develpment of therapeutics (Radare and Haenni, 1999). All DExH/D proteins characterized to date hydrolyse neucleoside triphosphates and, in most cases, this activity is stimulated by the addition of RNA or DNA. Several members of the family unwind RNA duplexes in an NTP dependent fashion in vitro (de la Cruz, et al., 1999 and Wagner, et al., 1998); therefore it has been proposed that DExH/D proteins couple NTP hydrolysis to RNA conformational change in complex macromolecular assemblies (Stanley and Guthric, 1998). Despite the central role of DExH/D proteins, their mechanism of RNA helicase activity remains unknown. It is shown that the DExH protein NPH-II unwinds RNA duplexes in a processive, unidirectional fashion with a step size of roughly one-half helix turn and that there is a quantitative connection between ATP and helicase processivity, thereby providing direct evidence that DExH/D proteins can function as molecular motors on RNA.

### SUMMARY OF THE INVENTION

The present invention provides a method for detecting the release of a single-stranded RNA from an RNA duplex which comprises: (a) admixing an RNA helicase with the RNA duplex under conditions permitting the RNA helicase to unwind the RNA duplex and release single-stranded RNA, wherein the RNA duplex comprises a first RNA having a first label attached thereto and a second RNA, such first label being capable of producing a luminescent energy pattern when the first RNA is present in the RNA duplex which differs from the luminescent energy pattern produced when the first RNA is not present in the RNA duplex, (b) subjecting the admixture to conditions which permit (i) the RNA helicase to unwind the RNA duplex and release single-stranded RNA, and (ii) the first label to produce luminescent energy; and (c) detecting a change in the luminescent energy pattern produced by the first label so as to thereby detect release of single-stranded RNA from the RNA duplex.

### BRIEF DESCRIPTION OF THE FIGURES

FIG. 1. Substrate design and structure of the U1A binding site. A. Sequence and secondary structure of the U1A binding site in the U1A mRNA 3′ UTR (21). Orange and blue letters correspond to the nucleotides retained in the substrates and present in the structure (panel D). B. Substrate. Colored letters represent nucleotides retained in the WT U1A binding site, black letters correspond to the nucleotides added as described in the text. The duplex regions are identical to sequences included in constructs used to study the structure of the complex (9). The 24 nucleotide single strand overhang (AN₂₂U-3′) has the sequence 3′-UACAGUAACUACGACAAUCAUGCA. C. Blunt-end control RNA. D. Structure of the U1A RNA complex as determined by NMR ((12), PDB accession # 1DZ5). The two protein units are drawn as a transparent surface with ribbons representing the backbone. The two RNA strands are drawn as ladder with the sticks corresponding to the bases and the ribbon corresponding to the backbone. The location of the 3′ end with the single-strand overhang on the RNA substrate is indicated.

FIG. 2. U1A binding to RNA substrate and its effects on unwinding. A. U1A binding to substrate RNA (FIG. 1B). Radiolabeled substrate (1 nM) was combined with U1A (10 nM) in a buffer containing 40 mM Tris/HCl (pH 8.0) and 4 mM MgCl₂ (in a final volume of 10 μL). After incubating at room temperature for 5 min, glycerol was added (8% v/v final) and the mixture was subjected to 8% native PAGE at 4° C., running at 10V/cm. Bands were visualized by a PhosphorImager. Species corresponding to free substrate RNA, bound U1A monomer, and bound U1A dimer are indicated at left. The asterisk represents a radiolabel. Left lane: RNA substrate bound to U1A; right lane: free substrate. B. The effect of U1A binding on duplex unwinding. Reactions were performed at room temperature for 5 min with 1 nM RNA substrate and 20 nM NPH-II in a buffer of 40 mM Tris/HCl (pH 8.0), 4 mM MgCl₂, and, if applicable, 3.5 mm ATP (10 μL final volume). Where present, the U1A concentration was 10 nM. Substrate and U1A were pre-incubated for 5 min at room temperature.- NPH-II was added and incubated for 5 more minutes. The reaction was then started by addition of ATP. Reactions were quenched by adding 10 μL of a solution containing 25 mM EDTA, 0.4% SDS, 0.05% BPB, 0.05% XCB, and 10% glycerol. Mixtures were subjected to 15% native PAGE which was run at room temperature at 20V/cm. Lanes from left to right. Unwinding reaction without ATP; NPH-II unwinding reaction; unwinding in the presence of 10 nM U1A; boiled substrate. Unwound and duplex species are indicated by the cartoons at right. C. U1A binding to the blunt end control RNA. Binding reactions were performed as described and shown in panel A. D. Unwinding reactions with the blunt end control RNA. Lanes correspond to those in panel B.

FIG. 3. Active displacement of U1A by NPH-II. Dissociation experiments were conducted with 1 nM RNA substrate and 10 nM U1A in a buffer of 40 mM Tris/HCl (pH 8.0), 4 mM MgCl₂ at 23° C. (final volume, 40 μL)- Reactions were performed as described in FIG. 2, except that they were initiated by addition of ATP (3.5 mM final concentration) and U1A trap (200 nM final concentration (16)), as indicated. Aliquots (6 μL) were withdrawn at 0, 1, 4, 8, 12, and 20 minutes, mixed with 2 μL of 100 mM EDTA and 2.5 μM NPH-II trap (which serves to capture dissociated NPH-II (16)) in 30% glycerol. Each aliquot was then loaded immediately on a 8% native polyacrylamide gel, which was run at 4° C. at 10V/cm. Bands were visualized by PhosphorImager. U1A-bound and free RNA as well as unwound and duplex RNA species are indicated by the cartoons at left (D: bound U1A dimer, M: bound U1A monomer, F: U1A free duplex substrate, U: unwound substrate). A. Release of U1A upon addition of ATP (initiated by adding ATP together with U1A trap). B. Release of U1A upon addition of NPH-II without ATP (initiated by adding only U1A trap). C. Release of U1A in the presence of ATP and NPH-II (initiated by adding ATP together with U1A trap) D. Trapping control: U1A trap is added together with RNA substrate to assess trapping efficiency. Aliquots were removed and treated as described above. E-H. Same reactions as above, but with blunt-end control duplex.

FIG. 4. Mechanism of U1A displacement by NPH-II. A. Timecourse of U1A displacement and substrate unwinding with and without NPH-II trap. Reactions without NPH-II trap were conducted as described in FIG. 3. The reaction with NPH-II trap was initiated by adding a combination of ATP (3.5 mM final concentration), U1A-trap (200 nM final concentration) and NPH-II trap (500 nM final concentration). Aliquots were withdrawn at the times indicated in the plots (panel B and C). U1A bound, free substrate, and unsound substrate species are indicated by the cartoons at left (Bound: U1A dimer and monomer, Free: non-bound and non-unwound substrate; U: unwound substrate) B. Plot of reaction without NPH-II trap for Bound, Free, and Unwound substrate (normalized (22) multiple cycle conditions). The monomer and dimer forms of bound U1A decayed with roughly the same rate and were therefore combined as the bound fraction (B). Solid lines are the simulated fits of the data based on the reaction mechanism described below (19), using the emipirically-determined rate constants (panel D). C. Plot of reaction with NPH-II trap (single-cycle conditions). Solid lines are the best fit to the integrated rate laws derived from the mechanism below (17). D. Kinetic mechanism of UIA displacement and unwinding by NPH-II. The red circle represents NPH-II, and the blue elipsoids represent U1A. Rate constants were calculated according to integrated rate laws describing single-cycle reaction kinetics (17), using three different timecourses (Panel C). Abbreviations: NSUP: NPH-II-substrate-U1A-complex prior to reaction initiation, NSUI: NPH-II-U1A-substrate-complex after the first rate limiting step; SU substrate-U1A-complex (after NPH-II dissociation); NS: NPH-II-substrate-complex (after U1A displacement); S: substrate (after U1A displacement and NPH-II dissociation); P: unwound product. Note that at the end of reaction, NPH-II dissociates rapidly and irreversibly from the substrate and that fraction of substrate bound to U1A consists of the species NSUₚ, NSUᵢ and SU. The fraction of free substrate comprises NS and S.

FIG. 5

Kinetic approach to probe processivity and directionality of the unwinding reaction. Unwinding of a dsRNA is the reverse reaction of the association of two complementary ssRNAs. Thus, unwinding occurs only when so many basepairs are disrupted that no nucleation locus can form. Practically, that means that unwinding is only observed, when less then 4 or 3 basepairs remain in the duplex. This emphasizes that by monitoring a duplex unwinding reaction one actually monitors the very last event of the strand separation process.

A processive reaction occurs in distinct consecutive steps, therefore, there are reaction intermediates (I) until the reaction product (P) is formed. For a non-processive reaction (for instance if the unwinding occurs in one power stroke) there are no intermediates. Thus, the experimental rationale for probing processivity is to determine whether there are reaction intermediates or not. If the reaction proceeds in distinct consecutive translocation steps, the translocation is rate limiting for the unwinding reaction, and the rate constants (kₜᵣ) of these translocation steps are of similar or equal size, intermediates (I) would accumulate. This would be reflected in a lag phase in the unwinding timecourse of the product (P) formation. With increasing duplex length more reaction intermediates would appear and timecourses would display a greater lag phase with increasing duplex length. The number of intermediates (I_{1 . . . n}) is indicative for the number of kinetic steps that the helicase takes to unwind a duplex of given length. More intermediates cause also more dissociation “events”, provided that these intermediates are susceptible to dissociation. This is determined by the ratio of the translocation rate constant (kₜᵣ) to the respective dissociation rate constant (k_{di}) (Ali and Lohmann, 1997). If a measurable fraction of protein dissociates during each unwinding step, the reaction amplitude should decrease with increasing duplex length. This should also occur when the translocation is not rate-limiting for the unwinding reaction. However, in that case the duplex length should not affect the reaction rate. If the reaction is not processive, there should be under no conditions a sensitivity of either reaction rate or amplitude to the length of the duplexes.

Probing the processivity is the prerequisite for testing the directionality of the unwinding, since only a processive reaction can be directional. The directionality can be directly tested by monitoring at least one reaction intermediate and the reaction product at the same time under conditions where the translocation limits the unwinding reaction. The experimental conditions for probing processivity and directionality have to be chosen in a way, that all RNA is complexed with protein at the reaction start and that all dissociation of the protein off the RNA substrate is irreversible.

FIG. 6

Unwinding of various RNA substrates by NPH-II. Unwinding reactions were performed in a reaction buffer comprising 30 μL volume(40 mM Tris.Cl, pH 8.0, 2 mM DTT, 20 mM NaCl, 3 mM MgCl₂, 3 mM ATP, 16 nM NPH-II, 3 nM RNA substrate) at 23° C. Protein, RNA substrate, and MgCl₂ were incubated in reaction buffer prior to the reaction for 7 min. The reaction was started by addition of ATP. Aliquotes were withdrawn at appropriate times and added to two volumes quenching buffer (25 mM EDTA, 0.4% SDS, 10% glycerol, 0.05% BPB, 0.05% XCB). This mixture was immediately cooled on ice. After the last aliquot was taken, the samples were applied to 15% native PAGE.

FIG. 7

Effect of trap RNA on unwinding reactions. (A) Since reactions are performed with preannealing of protein and RNA prior to the reaction, the protein-RNA complex [ES] is already formed at the reaction start. As protein dissociates from the RNA substrate during the unwinding reaction, re-binding of the protein and multiple reaction cycles can occur. Binding (“trapping”) free protein during the course of unwinding prevents re-binding during the reaction. Complete “trapping” of all free protein is ensured by using a large excess of RNA (trap RNA) as compared to the duplex substrate. (B) Effect of trap addition on reactions with LS83 and LS36. Reaction conditions were as described in FIG. 6, except that the ATP was pre-mixed with the trap RNA (39-mer RNA oligo) and the reaction was started with this ATP/trap mixture. The trap concentration in the reaction was 600 nM which is an 200 fold excess over the substrate. Higher concentrations of trap did not change the observed timecourses. The substrate was bound to 100% of the protein prior to the reaction as verified by gel shift analysis of the RNA protein complexes (not shown).

FIG. 8

Monitoring duplex unwinding by fluorescence energy transfer. (A) Both strands of the duplex are labeled with a fluorescence transfer donor-acceptor-pair of fluorescent dyes. The top strand is labeled with flourescein (donor) at the 3′-end and the bottom strand is labeled with rhodamin (acceptor) at the 5′-end. The attached fluorescein is excited at 492 nm. Due to the proximity of the acceptor label in the duplex, the emission of the fluorescein label is quenched. Upon unwinding, both labels depart from each other, the quenching of the fluorescein label no longer occurs and an increase in the emission of the fluorescein fluorescence can be detected. (B) The unwinding reactions were performed as described in FIG. 7, but in a volume of 600 μL in a fluorescence cuvette. The fluorescence intensity was measured at defined intervals (small points). The resulting curve was corrected for the reaction amplitude derived by gel shift measurements of identical reactions and both, the derived fluorescence curve and the measurements from gel shift experiments (large points) are overlaid in the plots. Reactions were performed with US18 (18 bp duplex region) and US36 (36 bp duplex region).

FIG. 9

Effect of ATP concentration on the unwinding reaction. Unwinding reactions with LS36 were performed as described in FIG. 7 (pre-annealing, trap addition).

The plot shows the final amplitude of the reaction versus the respective ATP concentration. Amplitude values were determined out of 8 aliquots taken from the reaction.

FIG. 10

Effect of Mg²⁺ substitution on the unwinding reaction. Timecourses with US36 were measured as described in FIG. 7 (trap RNA, pre-annealing), except that 3 nM Mg²⁺ (A) was substituted with 3 mM Mn²⁺′ (B) and 3 mM Co²⁺ (C). The left panels show the gel printouts of the reactions that are plotted on the right panels as fraction unwound substrate versus time.

FIG. 11

Effect of the duplex length on unwinding reactions in the presence of Co²⁺. Unwinding reactions were performed with US36 (36 bp duplex region) and US18 (18 bp duplex region) under the conditions described in FIG. 10. The solid lines represent fits for a consecutive 2 step reaction with equal rate constants (Ali and Lohmann, 1997) for the 18 bp duplex and for a consecutive 4 step reaction with equal rate constants for the 36 bp duplex.

FIG. 12

Probing the directionality of the unwinding reaction by using multi-piece substrates. (A) Multi-piece substrates were generated by site directed processing of the top strand RNA with an engineered DNAzyme (Santoro and Joyce, 1997) and subsequent hybridization of the two “pieces” with the bottom strand. (B) Unwinding reactions of MPS 36/75 (36 nt oligo binds next to the single stranded overhang and 75 nt oligo binds adjacent to this strand) and MPS 83/28 (83 nt oligo binds next to the single stranded overhang and 28 nt oligo binds adjacent to this strand) in the presence of Mg²⁺ were carried out as described in FIG. 7. The fraction of the displaced pieces was calculated according to frac[N]ₜ=[Nₜ/(Nₜ+Mₜ+Sₜ)]*[(N_{∞}+M_{∞}+S_{∞})/N_{∞}], where frac[N]ₜ is the fraction of the respective “piece” at the time t; Nₜ is the intensity of the band of the “piece” N at the time t; Mₜ is the intensity of the band of the “piece” M at the time t; Sₜ is the intensity of the not unwound substrate band at the time t; N_{∞} is the intensity of the band of the “piece” N after heat denaturation; M_{∞} is the intensity of the band of the “piece” M after heat denaturation and S_{∞} is the intensity of the not unwound substrate band after heat denaturation. S_{∞} is, however, usually zero. The solid lines represent a fit of the timecourse to a rate law describing a sum of two first order reactions. (C) Reactions of MPS 36/75 and MPS 83/28 in the presence of Co²⁺ were performed as described in FIG. 10. The fraction of the displaced pieces were calculated as described above. The solid lines represent fits of the timecourses to rate laws describing a consecutive 4 step reaction for T36, a 12 step reaction for T75 and T28 and a 9 step reaction for T 83, using equal rate constants for the steps, respectively.

FIG. 13

Kinetics of duplex unwinding. Reactions in the presence (open circles) and absence (filled circles) of trap RNA with a 36-bp substrate (a) and a 83-bp substrate (b). The 83-bp substrate is an extension of the 36-bp substrate. The fraction of unwound substrate (indicated on the y axis) was fitted to the integrated first-order rate law using Kaleidagraph software (Abelbeck) . Rate constants (k) and reaction amplitudes (A) in the absence of trap RNA: k=3.5±0.1 min⁻¹, A=0.96, 36-bp substrate; k=3.4±0.1 min⁻¹, A=0.97, 83-bp substrate. In the presence of trap RNA: k=3.6±0.2 min⁻¹, A=0.77, 36-bp substrate; k=3.6±0.3 min⁻¹, A=0.52, 83-bp substrate. Variances are standard deviations from the fit.

FIG. 14

Estimation of the unwinding step size. Time courses of substrates containing duplex regions of 12 (open squares), 18 (filled squares), 24 (open circles) and 36 (filled circles) bp unwound by NPH-II in the presence of 4 mM CoCl₂. Substrates contain a 35-U single-strand overhang 3′ to the duplex region. Step size and the unwinding (translocation) rate constant were calculated were calculated from at least two independent time courses for each duplex which are overlaid on the plots. Solid lines are best fits to the kinetic model resulting in an unwinding step size of 6 bp and an average unwinding rate constant of k_{g}=13.4±0.8 min⁻¹.

FIG. 15

Probing helicase directionality with multipiece substrates (MPS) a, Design of the MPS b. Unwinding of MPS in 4 mM MgCl₂ and trap RNA. Unwinding rate constants were similar for all pieces (k=3.3±0.5 min⁻¹); reaction amplitudes (A) were MPS 36/75, T36 A=0.74; T75, A=0.32; MPS 83/28, T83, A=0.43; T28, A=0.27. c. Unwinding of multipiece substrates in 4 mM CoCl₂ and trap RNA. Kinetic data were analysed as described in FIG. 6, which resulted in average values of k_{g}=12.4±2.1 min⁻¹ and a step size of 6 pb, consistent with the values obtained for oligomers shown in FIG. 6.

FIG. 16

Processivity of NPH-II a, ATP dependence of processivity in 4 mM MgCl₂ (filled circles) and 4 mM CoCl₂ (open circles). The solid line is a hyperbolic fit: P=P[ATP]/(Z+[ATP]), where P_{∞}=k_{U[s}}/(k_{U[s}}+k₈) reflects the processivity at ATP saturation; Z is the ATP concentration where P=P_{∞}/2. In 4 mM CoCl₂, P_{∞}=1.00±0.03 and Z=0.50±0.06 mM. In 4 mM MgCl₂, P_{∞}=0.99±0.01 and Z=0.14±0.01 mM. Processivities were determined from the reaction amplitudes of four duplexes (12, 18, 24 and 36 bp, FIG. 6) at the ATP concentrations indicated. Amplitudes were obtained from at least three independent experiments. Processivity was calculated by fitting plots of amplitude at constant ATP concentration versus duplex length to Equation 2 using a step size of m=6 (FIG. 6). b, c, Dependence of reaction amplitude on ATP concentration for substrates with duplex regions of 12 (filled circles), 18 (open circles), 24 (filled squares) and 36 (open squares) basepairs in 4 mM MgCl₂ (b) and 4 mM CoCl₂ (c). Each reaction amplitude was determined in triplicate and the deviation from these independent measurements is indicated by the error bars. The solid lines are fits to equation 1, where k′_{d} was assumed to equal Kₘ from multiple turnover ATPase measurements (1.1 mM in Mg²⁺ and 3.5 mM in Co²⁺, (Shuman, 1993; Gross and Shuman 1996), the step size was 6 bp (FIG. 6), and kₚ/k_{U[S]} was allowed to flout. In both Mg²⁺ and Co²⁺ kₚ/k_{U[S]}=0.12.

FIG. 17

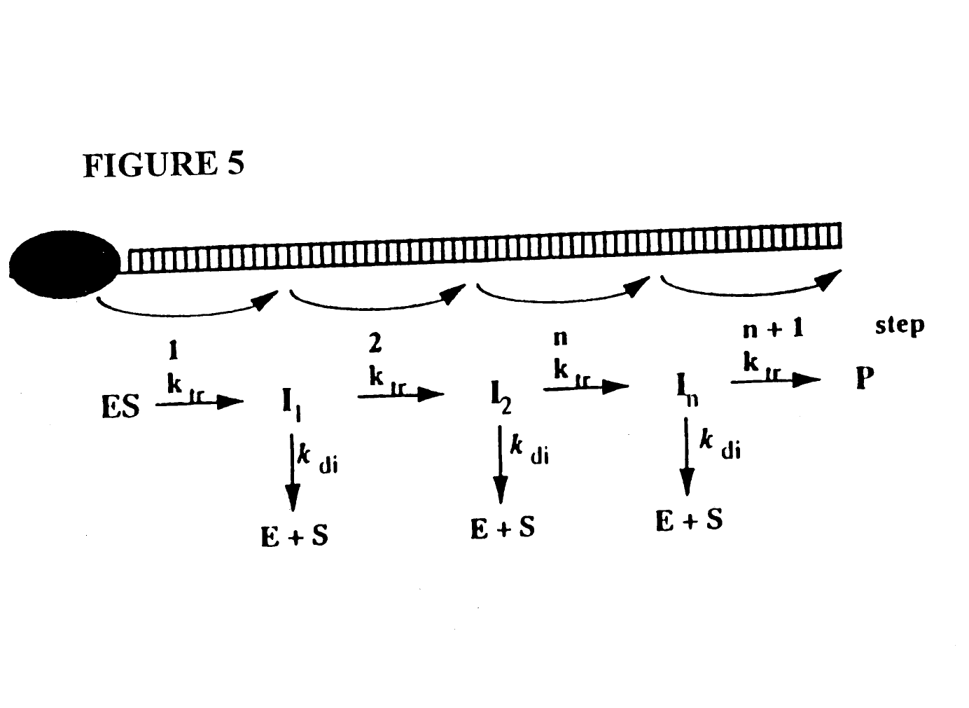

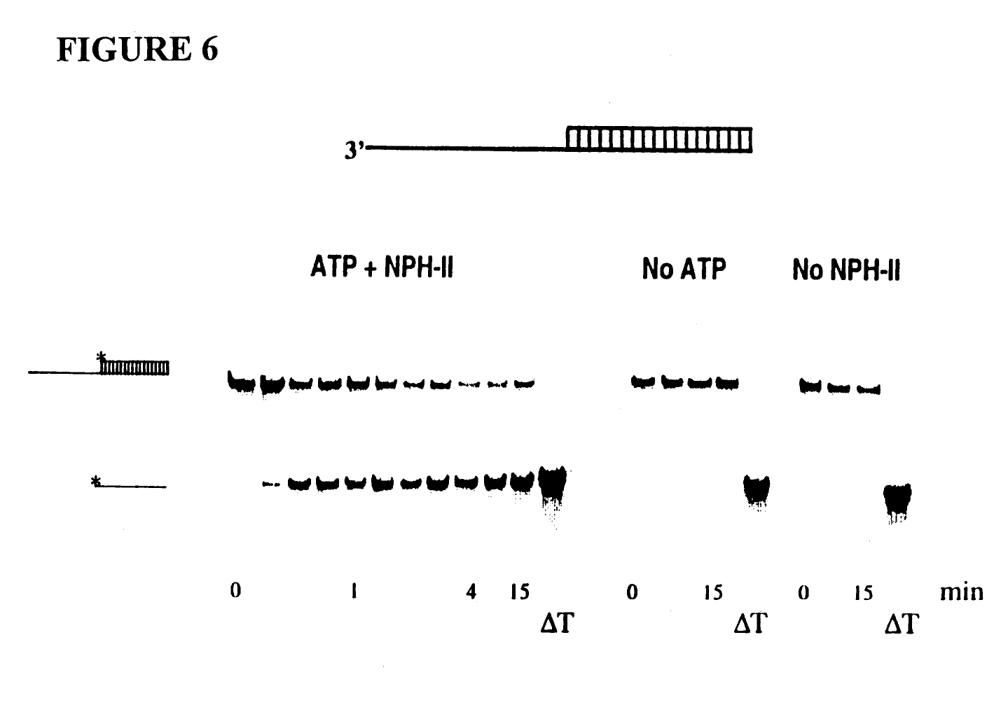

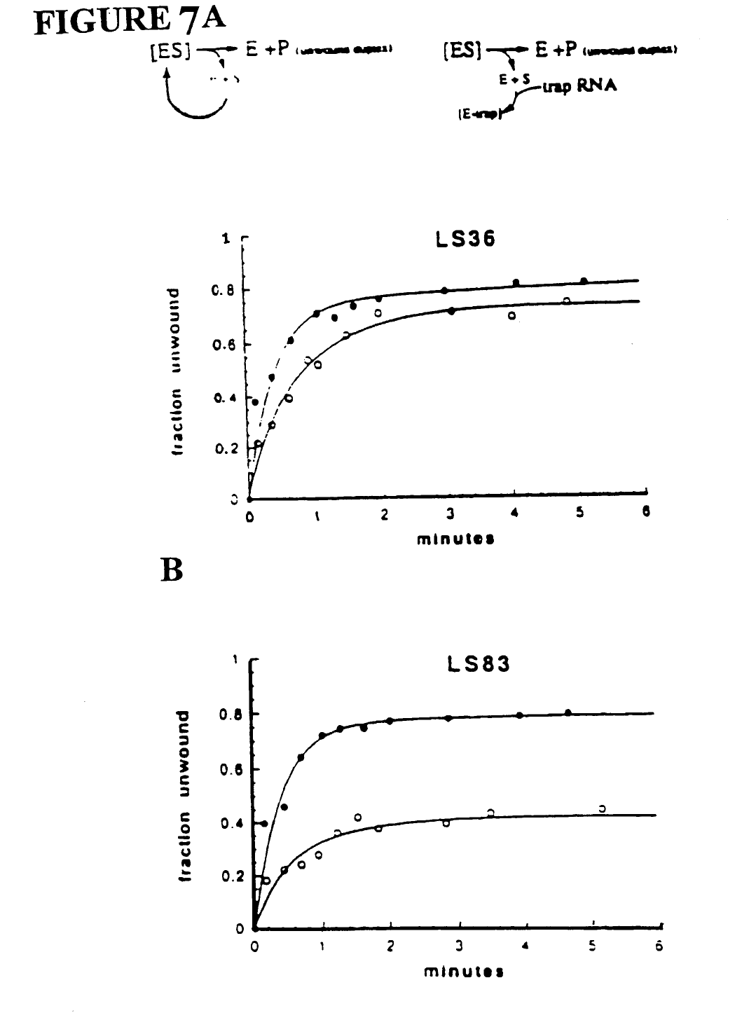

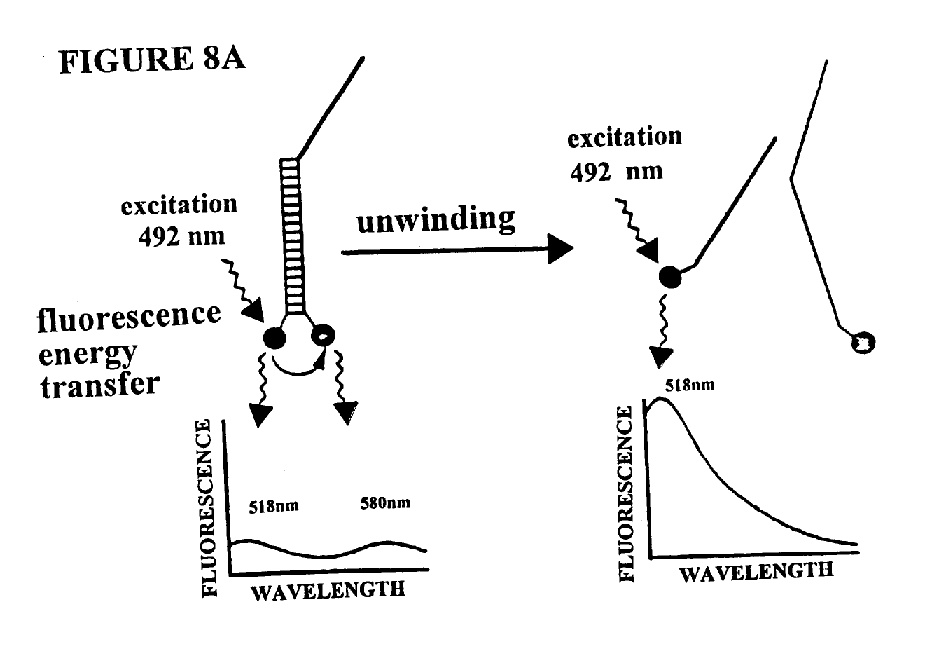

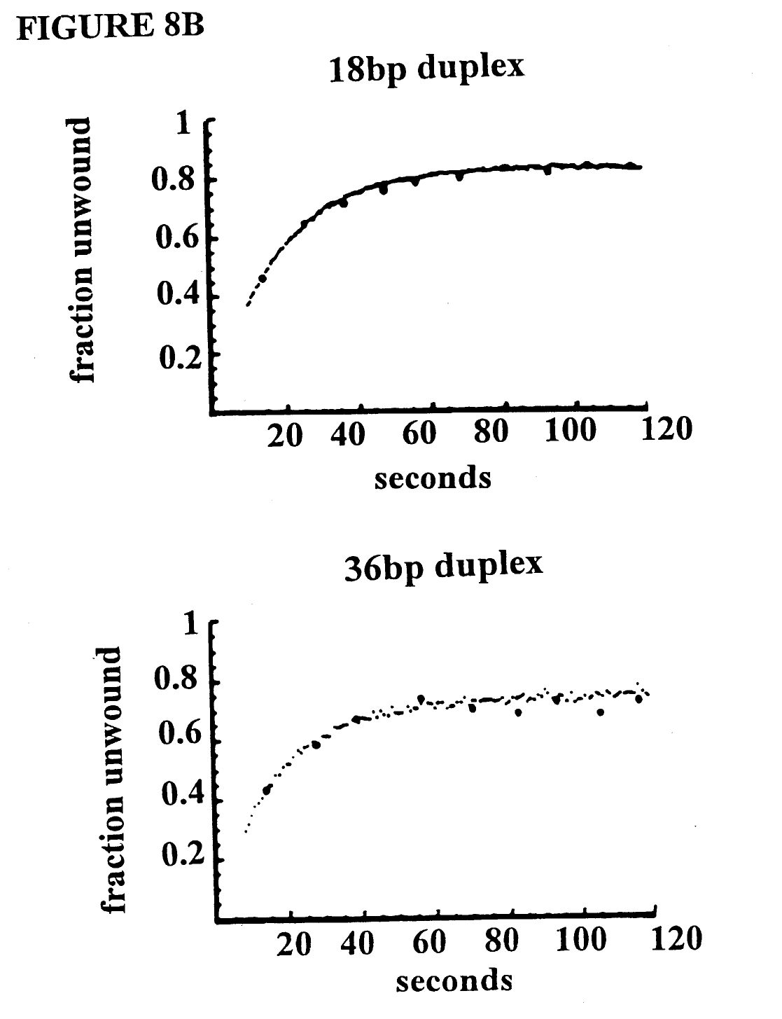

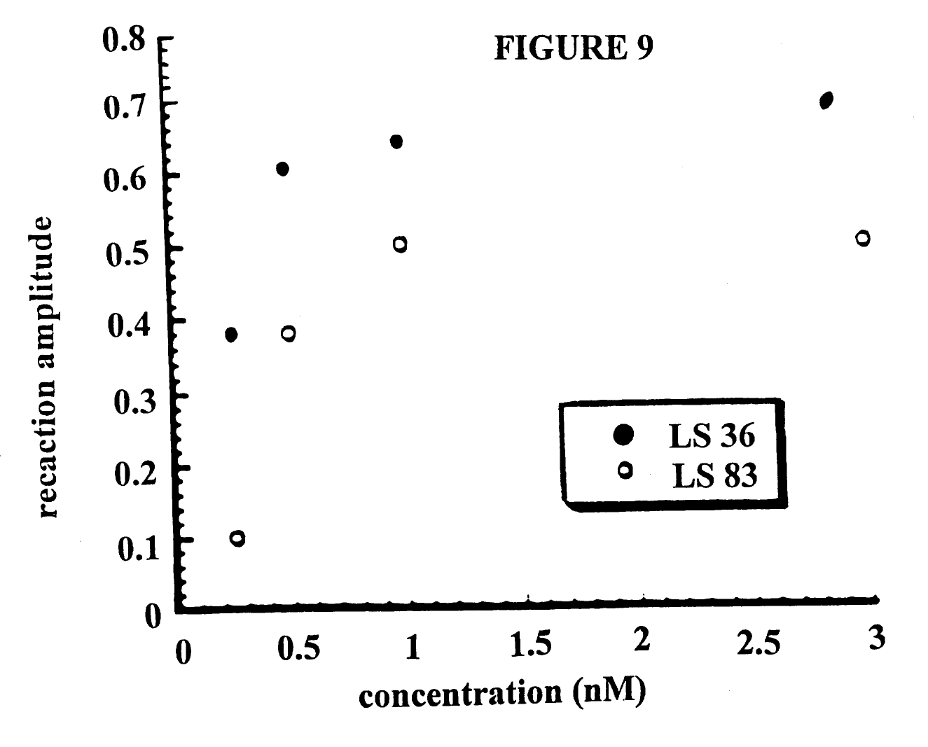

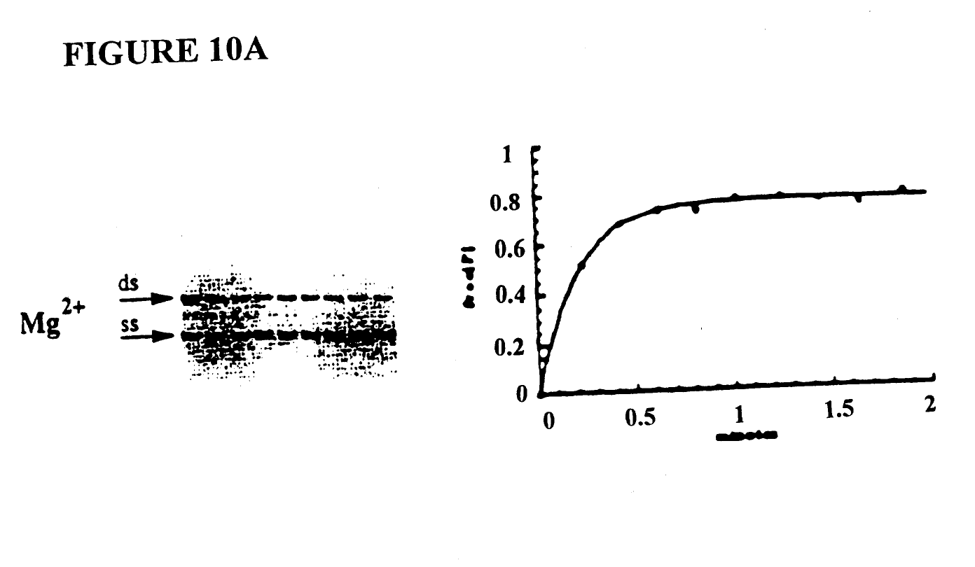

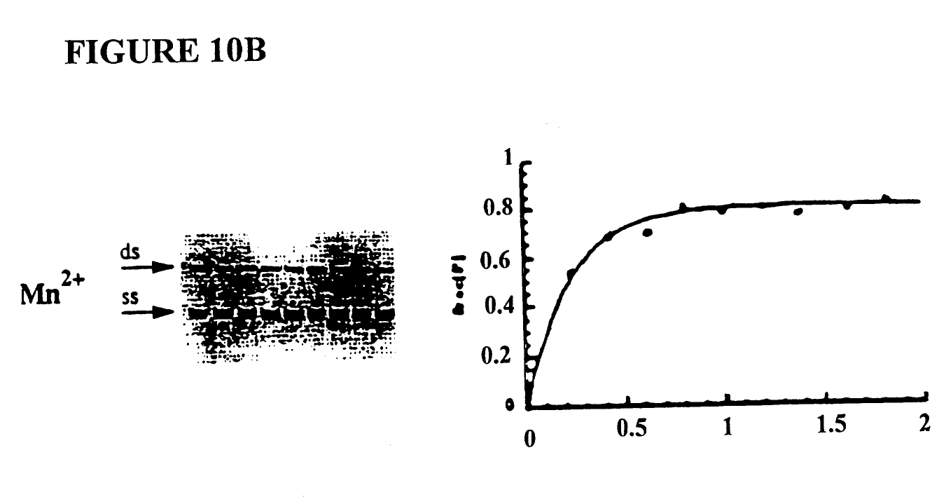

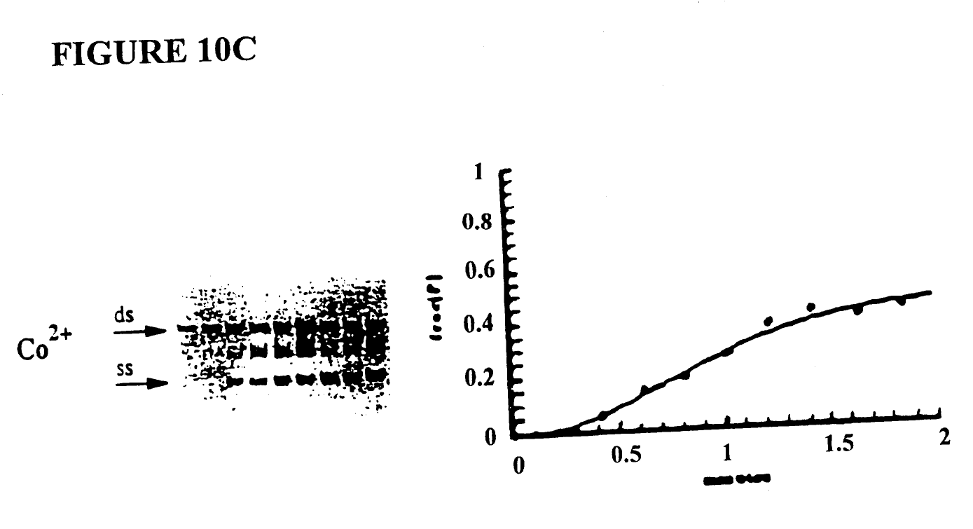

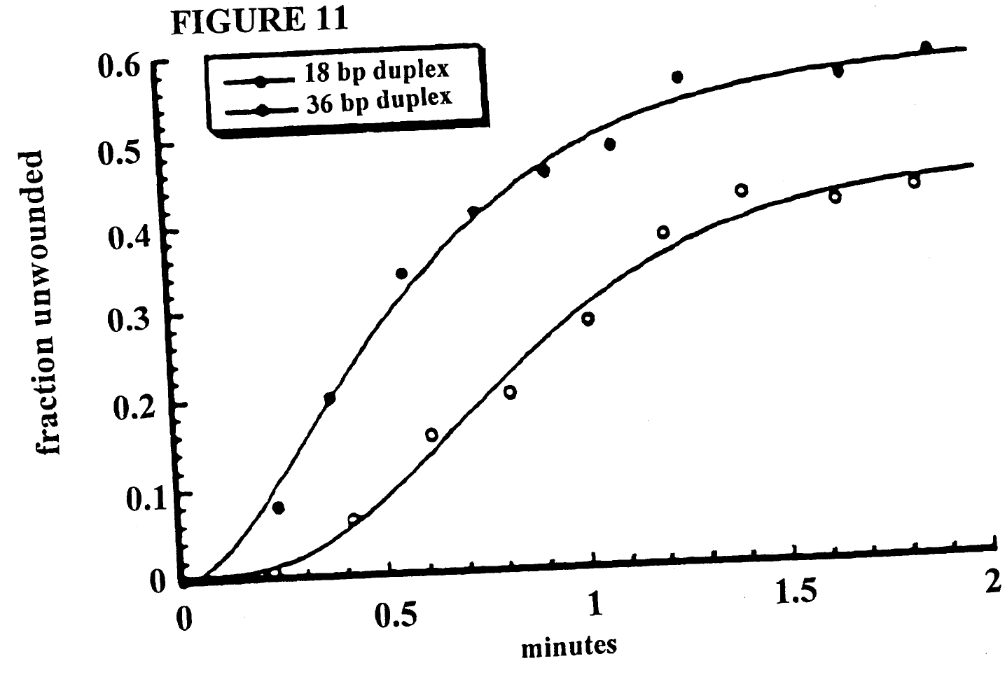

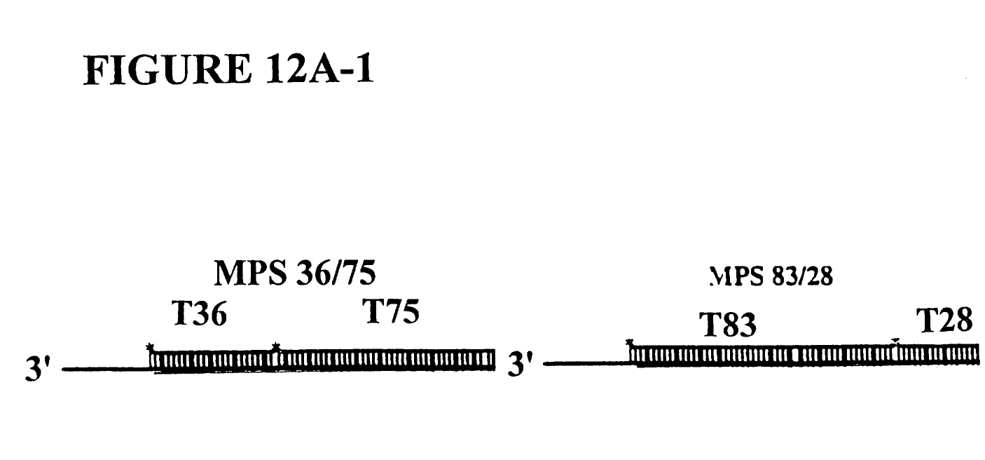

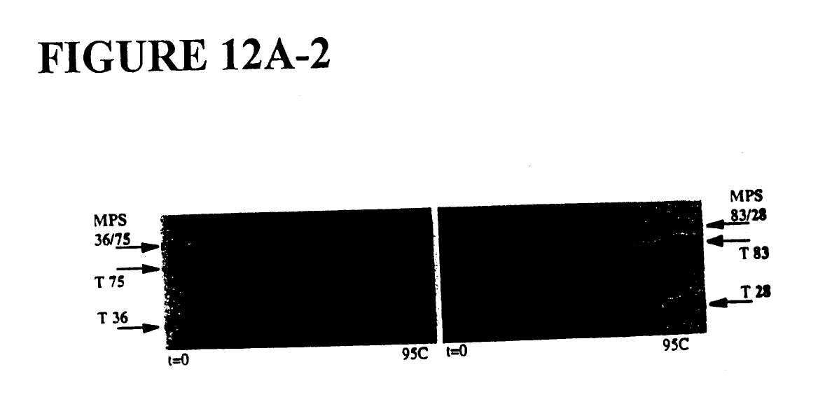

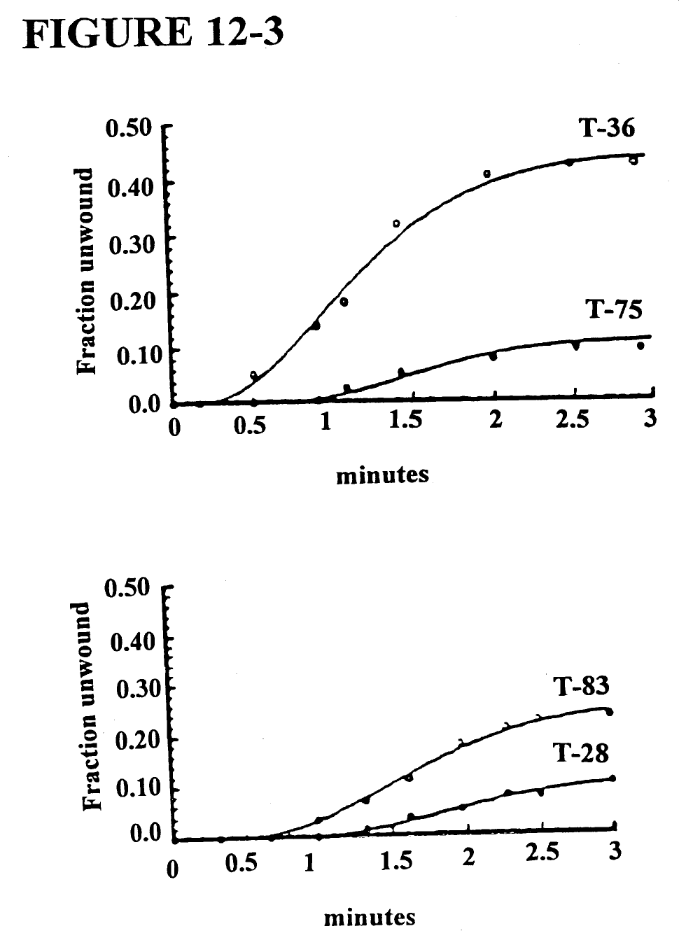

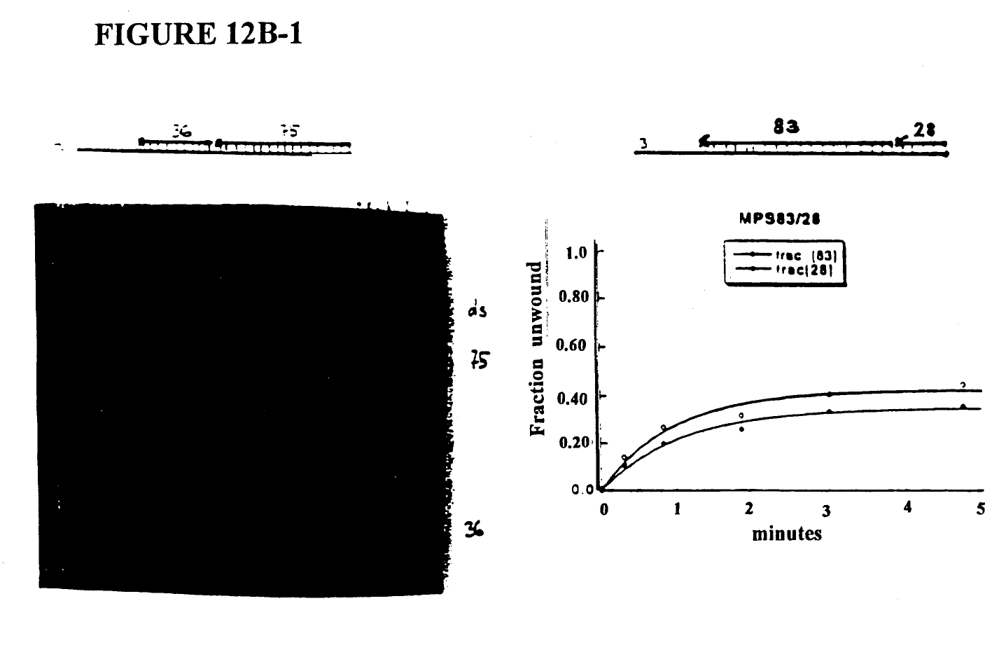

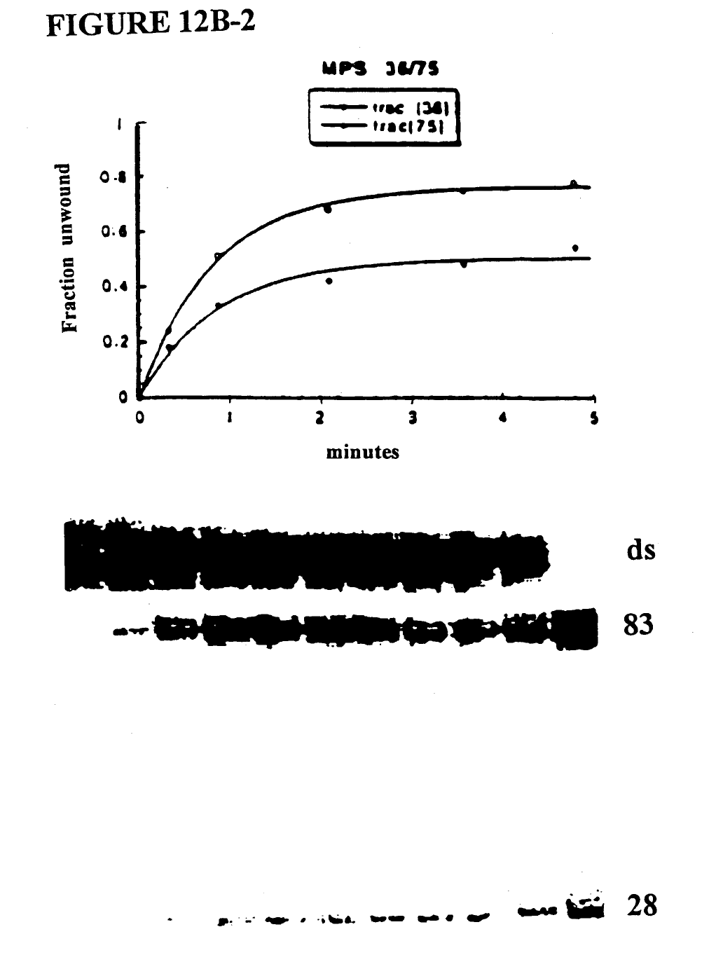

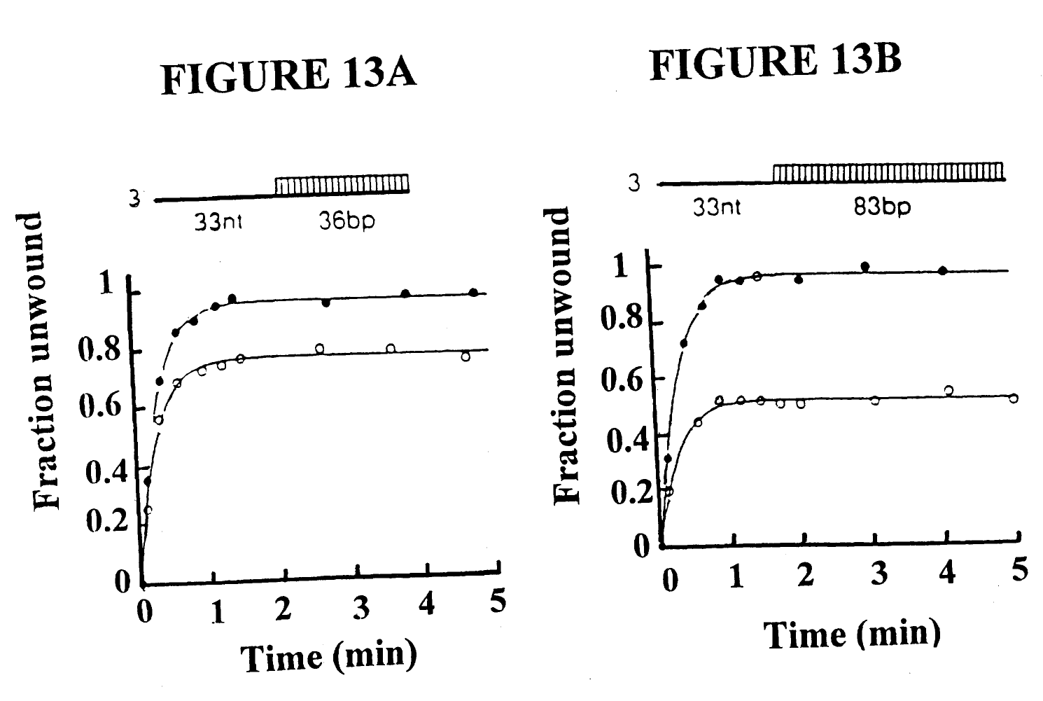

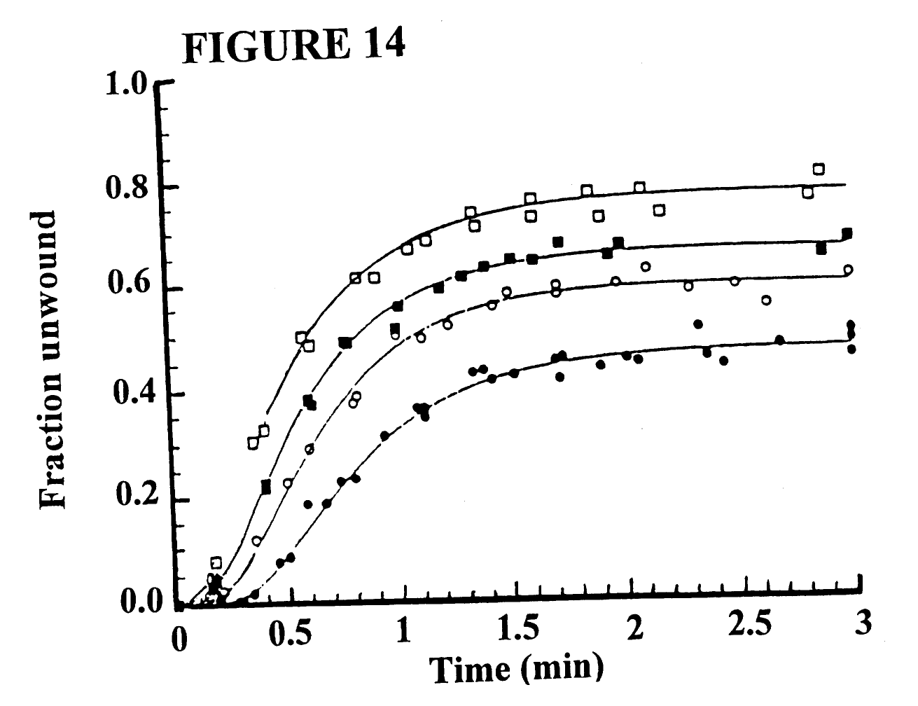

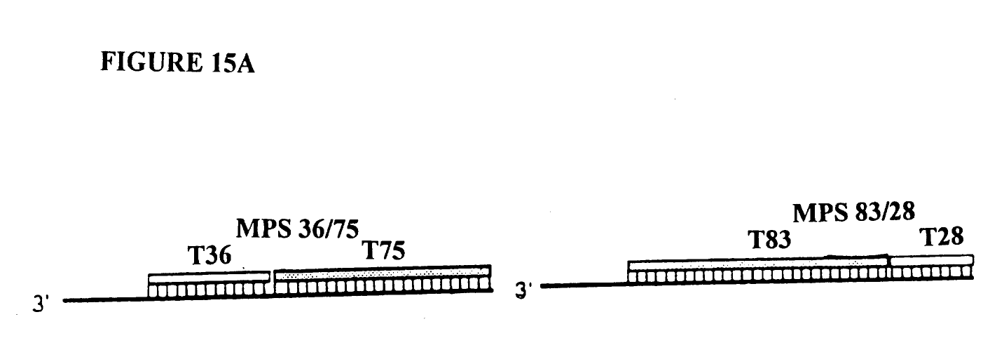

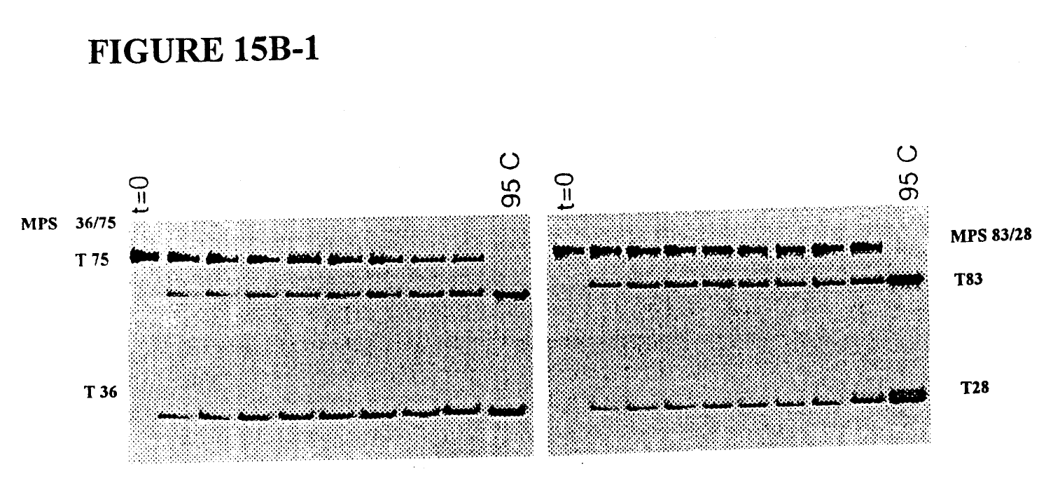

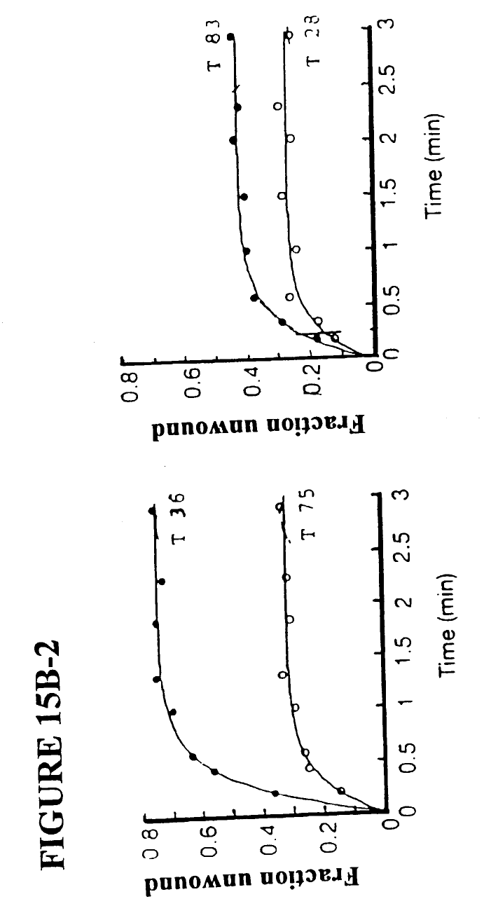

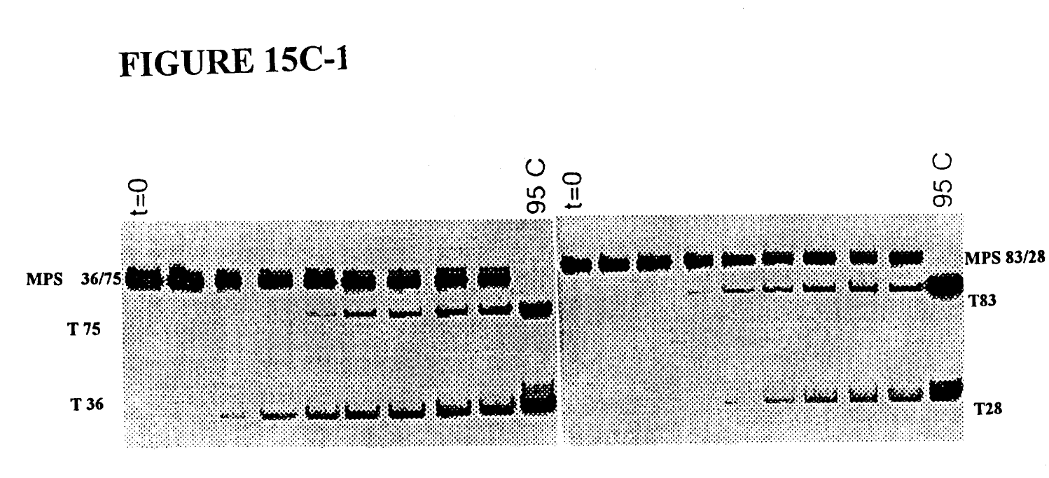

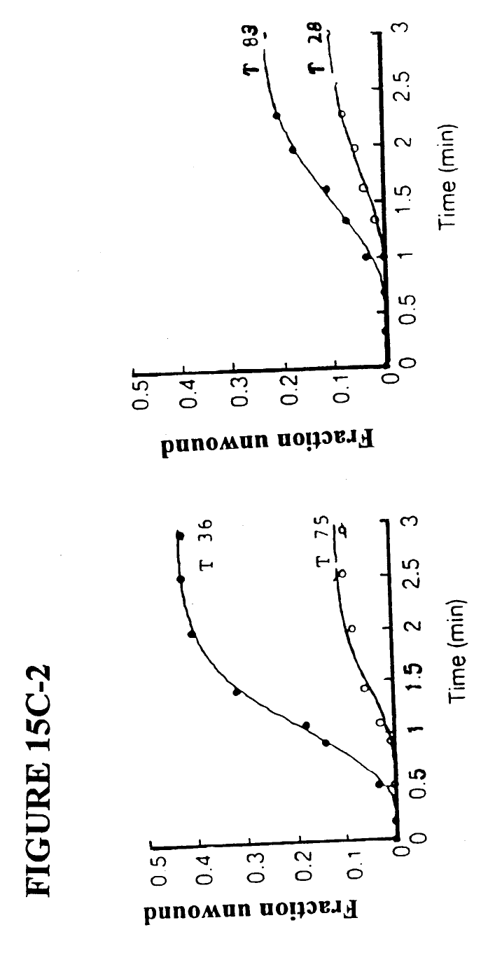

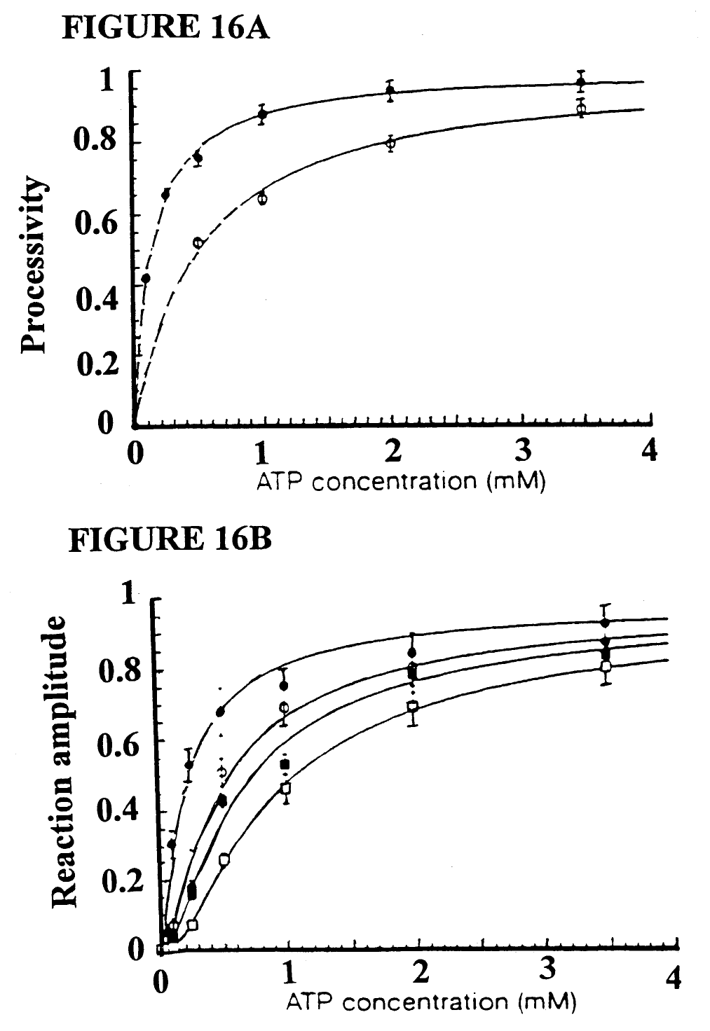

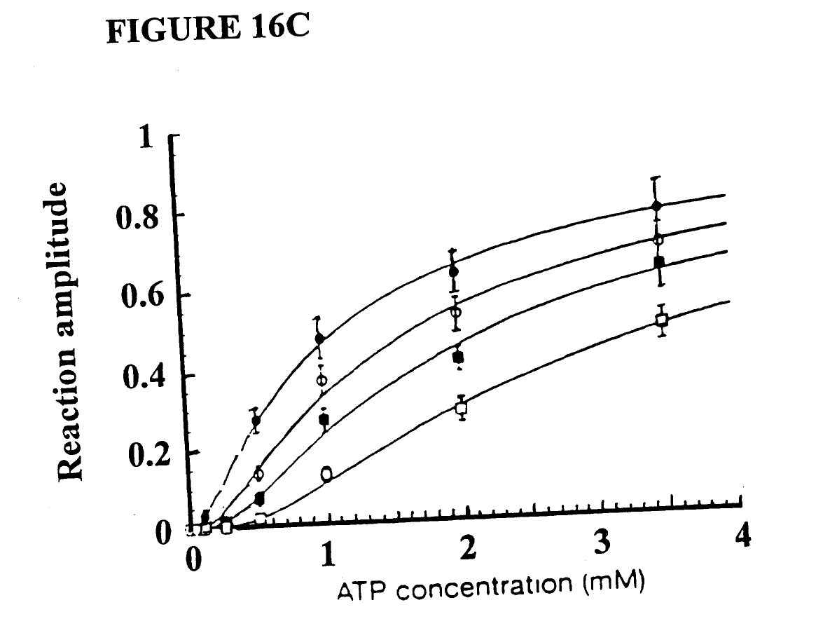

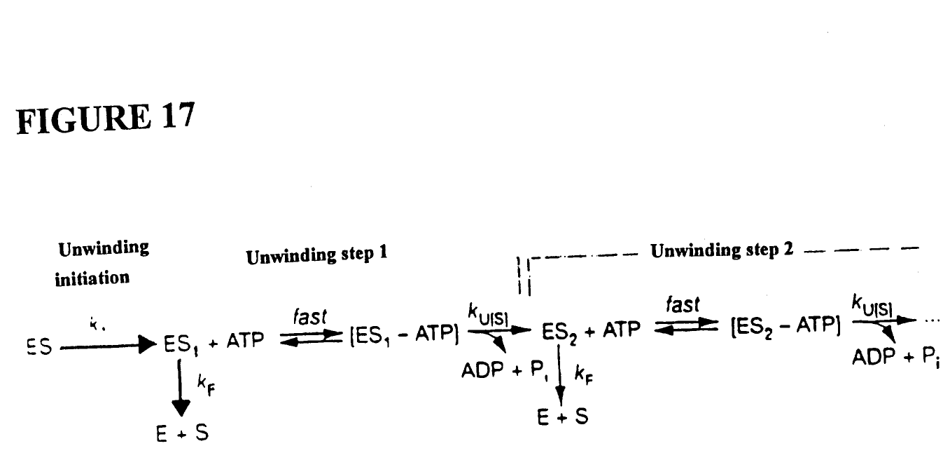

The kinetic scheme for RNA helicase activity by NPH-II. Unwinding initiation and two unwinding/translocation steps are depicted. ES₁ and ES₂ represent the enzyme substrate complex in non-ATP-bound states, while [ES₁-ATP] and [ES₂-ATP] represent ATP-bound states. Note that ‘ATP-bound’ indicates that ATP is bound but not hydrolysed, whereas ‘non-ATP-bound’ indicates that no nucleotide is bound or that ADP and/or inorganic phosphate are bound. The equilibrium between ATP-bound and non-ATP-bound states is fast compared with translocation with the unwinding rate constant k_{U[S}}.kₚ (Shuman, 1993; Gross and Shuman, 1996) is the rate constant for enzyme dissociation from subtrate in the ATP-free state, dissociation in the ATP-bound state is not significant (FIG. 8a) k₁ is the rate constant for the unwinding initiation step. Reactions were conducted in the presence of trap-RNA (FIG. 5), thus ES dissociation is irreversible.

### DETAILED DESCRIPTION OF THE INVENTION

The present invention provides an a method for detecting the release of a single-stranded RNA from an RNA duplex which comprises (a) admixing an RNA helicase with the RNA duplex under conditions permitting the RNA helicase to unwind the RNA duplex and release single-stranded RNA, wherein the RNA duplex comprises a first RNA having a first label attached thereto and a second RNA, such first label being capable of producing a luminescent energy pattern when the first RNA is present in the RNA duplex which differs from the luminescent energy pattern produced when the first RNA is not present in the RNA duplex, (b) subjecting the admixture to conditions which permit (i) the RNA helicase to unwind the RNA duplex and release single-stranded RNA, and (ii) the first label to produce luminescent energy, and (c) detecting a change in the luminescent energy pattern produced by the first label so as to thereby detect release of single-stranded RNA from the RNA duplex.

In accordance with the present invention conditions which permit the RNA helicase to unwind the RNA duplex and release single-stranded RNA preferably include, but are not limited to the presence of ATP and a divalent cation, which preferably may be Mg²⁺, Mn²⁺ or Co².

The foregoing method may be carried out in numerous ways which will be readily understood by those skilled in the art. In a presently preferred embodiment of the invention the method utilizes a second label attached to the second RNA such that the luminescent energy produced by the first label interacts with the second label to produce a characteristic pattern when the first and second RNA are present in an RNA duplex, but interacts differently or not at all when the RNA duplex unwinds. In one embodiment of the invention the first label is covalently attached at the 5′ end of the first RNA and the second label, if any, is attached at the 3′ end of the second RNA.

In accordance with the foregoing, embodiment the first and second label may comprise different fluorophors having the characteristic that the second label absorbs luminescent energy released from the first fluorophor when it is induced to emit luminescent energy by exposure to a particular type of radiation such as ultraviolet light of a defined wavelength. In one embodiment the first label is fluorescein isothiocyanate and the second label is rhodamine isothiocyanate.

As used herein luminescent energy includes but is not limited to fluorescence, phosphorescence, and chemiluminescence.

In the practice of the methods of the invention the compound capable of inhibiting unwinding activity of an RNA helicase may be a chemically synthesized substrate that preferentially binds over a endogenous substrate to the RNA helicase. In one embodiment of the invention the compound inhibits the accessibility of the ATP to the binding site on the protein thereby enabling the helicase to dissociate from the RNA at appropriate times. In another embodiment of the invention the compound induces a rate-limiting step by blocking and deblocking initiation of the RNA unwinding by the RNA helicase.

In the practice of the methods of the invention the compound capable of inhibiting the unwinding activity of an RNA helicase is present at a concentration capable of inhibiting the unwinding activity of an RNA helicase. Accordingly, the effective amount will vary with the helicase used.

This invention also provides a method of measuring the rate of release of a single-stranded RNA from an RNA duplex which comprises detecting whether the single-stranded RNA is released from the RNA duplex at predetermined time intervals according to the method and determining therefrom the rate of release of the single-stranded RNA from the RNA duplex.

Furthermore, this invention provides a method of determining whether a compound is capable of modulating the release of a single-stranded RNA from an RNA duplex by an RNA helicase which comprises detecting the release of the single-stranded RNA from the RNA duplex according to the method wherein the compound is added to the mixture of RNA duplex and RNA helicase.

Still further, this invention also provides effective amounts of compounds capable of inhibiting unwinding activity of an RNA helicase together with suitable diluents, preservatives, solubilizers, emulsifiers, adjuvants and/or carriers useful in treatment of viral RNA helicase or a viral Hepatitis C. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts) solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound. The choice of compound will depend on the physical and chemical properties of the compound capable of alleviating the symptoms of a viral RNA infection.

The present invention provides a method for detecting the DExH/D enzyme-facilitated release of a protein from a non-covalent complex formed between the protein and a nucleic acid molecule comprising (a) contacting the complex with a DExH/D enzyme under conditions permitting the enzyme to facilitate the release of the protein from the complex, with the proviso that the protein and/or nucleic acid molecule is labeled with a detectable marker which, upon release of the protein from the complex, provides a second signal differing from the first signal provided prior to such release; and (b) detecting the presence of the second signal provided by the detectable marker, thereby detecting the DExH/D enzyme-facilitated release of a protein from the complex.

In one embodiment, the protein only is labeled. In another embodiment, the nucleic acid molecule only is labeled. In a further embodiment, both the protein and nucleic acid molecule are labeled.

The present invention also provides a method for releasing a protein from a non-covalent complex formed between the protein and a nucleic acid molecule comprising contacting the complex with a DExH/D enzyme under conditions permitting the enzyme to facilitate the release of the protein from the complex.

The present invention further provides a method for determining whether a known protein is non-covalently bound to a nucleic acid molecule comprising (a) contacting a sample of the nucleic acid molecule suspected of having the protein bound thereto with a DExH/D enzyme under conditions permitting the release of the protein from the nucleic acid molecule if bound thereto, and (b) detecting the presence of any unbound protein in the sample, thereby determining whether the known protein was non-covalently bound to the nucleic acid molecule.

In these methods, the nucleic acid molecule can be DNA or RNA and can be single-stranded or double-stranded. The DExH/D enzyme can be any enzyme in this family, including, but not limited to, U1A, NPH-II, or any other RNP motif containing enzyme.

References relating to RNA-protein recognition include: Draper, D. E., (1999), Themes in RNA-protein recognition, J. Mol. Biol., 293(2):255-270; Cusack, S., (1999) RNA-protein complexes, Curr. Opin. Struct. Biol. 9(1): 66-73; Varani, G. and K. Nagai (1998), RNA recognition by RNP proteins during RNA processing, Ann. Rev. Biophys. Biomol. Struct. 27:407-445; and Frankel, A. D. and C. A. Smith, (1998), Induced folding in RNA-protein recognition: more than a simple molecular handshake, Cell 92(2): 149-151.

All embodiments of the instant invention as described elsewhere in this application are also envisioned, as applicable to the instant methods relating to the dissociation of protein/nucleic acid complexes.

This invention is illustrated in the Experimental Details section which follows. These sections are set forth to aid in an understanding of the invention but are not intended to, and should not be construed to, limit in any way the invention as set forth in the claims which follow thereafter.

Experimental Details

### Part I

The contents of this Part I were disclosed in E. Jankowsky, et al., (2001) “Active disruption of an RNA-Protein interaction by a DEXH RNA helicase”, Science 291: 121-125. (Jan. 5, 2001).

All aspects of cellular RNA metabolism and the replication of many viruses require DExH/D proteins that manipulate RNA in a manner that requires nucleoside triphosphates (NTPs). While DExH/D proteins have been shown to unwind purified RNA duplexes, most RNA molecules in the cellular environment are completed with proteins. It has therefore been speculated that DExH/D proteins may also affect RNA-protein interactions. Here we provide evidence that this is indeed the case. We demonstrate that the DEXH protein NPH-II from vaccinia virus can displace the protein U1A from RNA in an active, ATF dependent fashion. NPH-II increases the rate of U1A; dissociation by more then three orders of magnitude while retaining helicase processivity. This indicates that DExH/D proteins can effectively catalyze protein displacement from RNA and thereby participate in the structural reorganization of ribonucleoprotein assemblies.

Many DExH/D proteins hydrolyze nucleoside triphosphates (NTPs) in a reaction that is stimulated by nucleic acids and unwind RNA duplexes in an NTP-dependent fashion in vitro (1). DExH/D proteins are frequently part of large ribonucleoprotein (RNP) assemblies such as the spliceosome or viral replication machineries (2, 3). In some instances, DExH/D proteins have been shown to couple NTP hydrolysis to conformational changes in these complexes (4-6), and it is generally believed that this represents the predominant function of these enzymes in ribonucleoprotein assemblies (2).

Despite the importance of DExH/D proteins, little is known about mechanisms by which these enzymes effect the numerous conformational changes that occur in ribonucleoprotein machines. It has been demonstrated that DExH/D proteins can function as processive and directional molecular motors for unwinding regular RNA duplexes (7). Although unwinding of regular duplex RNA is clearly important in RNA metabolism, cellular RNA often has a more complex structure and, most importantly, is likely to be bound to proteins. This fact has prompted the attractive hypothesis that DExH/D proteins might not necessarily be “pure” RNA helicases; rather, they may also function to disrupt or re-arrange RNA-protein interactions (2). However, such activity by DExH/D proteins has never been demonstrated.

Here, we tested the ability of DExH/D proteins to displace proteins from RNA by investigating whether the DExH protein NPH-II from vaccinia virus can displace the protein U1A from a RNA substrate (8). NPH-II is an RNA helicase that unwinds RNA duplexes processively in the 3′ to 5′ direction with a kinetic step size of roughly one half helical turn (7). Use of a kinetically well characterized RNA helicase permits direct comparisons of the RNA unwinding and protein displacement activities. U1A is an ideal target protein because its RNA binding properties have been characterized (9). U1A binds RNA through an N-terminal RNP domain (10), which is the most common motif for mediating specific RNA-protein interactions (11). The active displacement of U1A is of particular interest because it is a constituent of the spliceosomal machinery and a feedback regulator of its own gene expression.

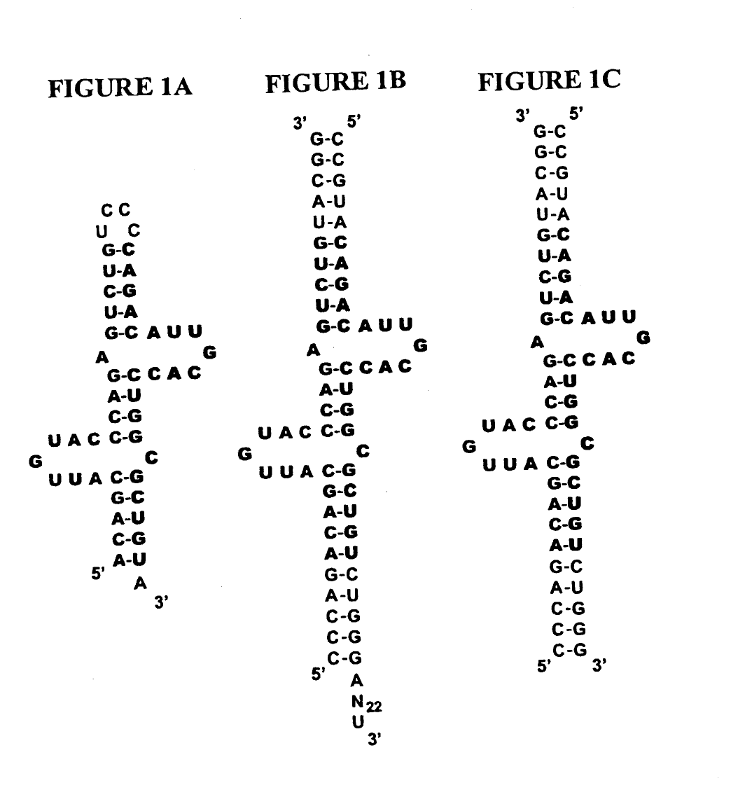

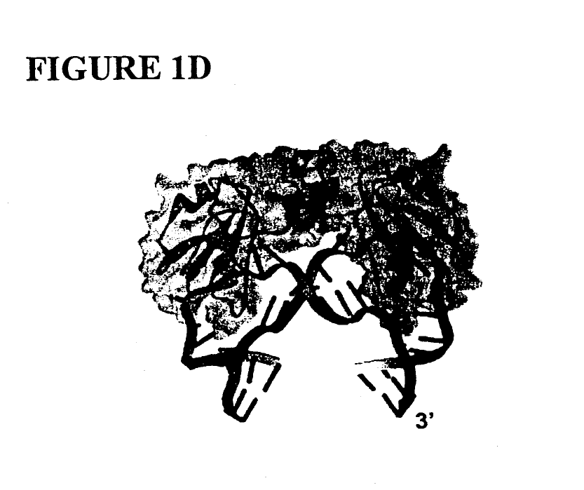

In order to simultaneously monitor U1A displacement and RNA helicase activity, a multifunctional RNA substrate was designed. The substrate contains the U1A binding site from the 3′-untranslated region (UTR) of U1A mRNA (FIG. 1A). U1A binds this motif as a dimer, interacting primarily with two asymmetric loop structures (12) that are imbedded within a set of duplex motifs ((11), FIG. 1D). To transform the U1A binding site into a helicase substrate, the hairpin loop was removed, and the flanking helical regions were lengthened (FIG. 1B). This type of extended, two-piece substrate for U1A binding has previously been shown to retain subnanomolar affinity for U1A binding (9). In order to promote high affinity NPH-II binding, a single strand 3′ overhang was appended to the duplex region (FIG. 1B, (13)). A blunt-ended control substrate was also synthesized, which contained the U1A binding site but lacked the single strand overhang (FIG. 1C)

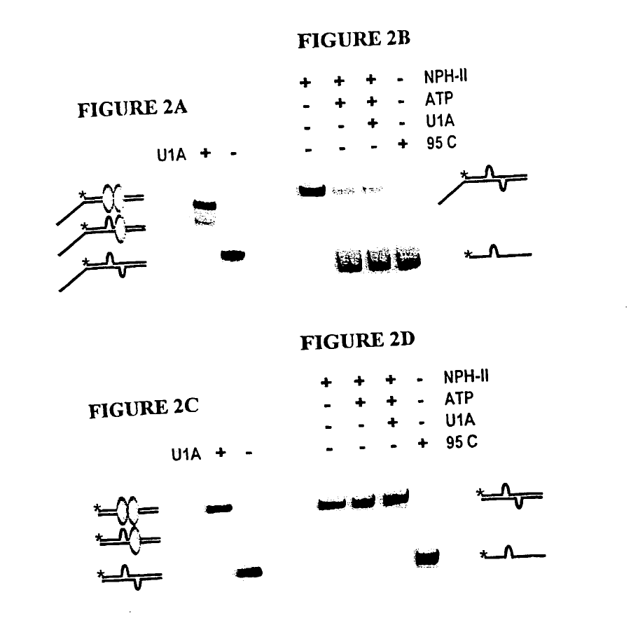

U1A bound to both substrate and control RNA with high affinity (FIG. 2 A, C), demonstrating that the base-paired extensions and, single strand overhang did not alter the binding of U1A.

The U1A binding site differs significantly from regular A-form helical geometry (FIG. 1D), and there is evidence that, even without bound U1A, the RNA is extensively bent (9). Despite this distortion in the RNA, NPH-II readily separated the two substrate strands in both the presence and absence of bound U1A (FIG. 2B). Most importantly, these findings establish that NPH-II can displace U1A. They also indicate that NPH-II can traverse loops and tolerate considerable bending in both substrate strands during duplex unwinding (14).

No unwinding was observed for the blunt-ended RNA substrate, regardless of whether U1A was bound (FIG. 2D). This provides two important controls: First, strand separation does not initiate at the internal loops and second, U1A binding does not provide additional opportunities for NPH-II to initiate unwinding.

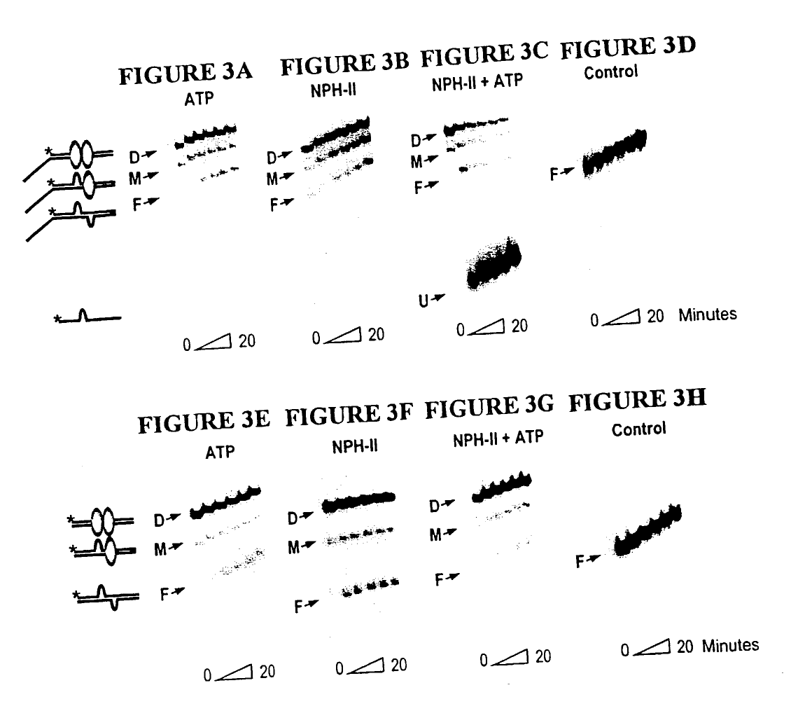

Next, it was important to distinguish whether NPH-II displaces U1A actively or in a passive manner. In the latter scenario, NPH-II would wait passively until U1A dissociates and then re-arrange the binding site such that U1A can no longer bind. In an active process, NPH-II would affect the kinetics of U1A dissociation from the RNA. We reasoned that it should be possible to distinguish both processes by measuring the effect of NPH-II action on U1A dissociation rates (FIG. 3).

The off-rate for U1A was measured by saturating the substrate with U1A and, after complex formation, adding a large excess of RNA that contains another high-affinity U1A binding site (15). This prevented U1A from re-binding the substrate once it detached and enabled us to monitor the rate of U1A release by gel-shift electrophoresis (FIG. 3).

Without NPH-II, roughly 15 percent of U1A dissociates from the substrate within 20 minutes, which corresponds to an off-rate of k_{off}˜10⁻² min⁻¹ (FIG. 3A) . In the presence of NPH-II, but without ATP, no unwinding is observed (compare FIG. 1B) and the off-rate was not significantly changed (FIG. 3B), which indicates that U1A is not displaced by mere binding of NPH-II to the substrate. However, adding both NPH-II and ATP resulted in a dramatically increased off-rate for U1A (FIG. 3C). After only 4 minutes, U1A was almost completely released from the substrate. This suggests a rate increase of several orders of magnitude and clearly demonstrates that NPH-II dissociates U1A from the substrate in an active, energy-dependent fashion.

The rate of U1A dissociation from the blunt-end RNA is similar to the rate of U1A dissociation from the helicase substrate in the absence of NPH-II (FIG. 3E), or in the presence of NPH-II without ATP (FIG. 3F). However, unlike the helicase substrate, NPH-II combined with ATP does not increase the rate of U1A dissociation (FIG. 3G). Thus, displacement of U1A by NPH-II is not caused by the structural peculiarities of the U1A binding site, but rather depends on binding of NPH-II to the single-strand overhang of the substrate.

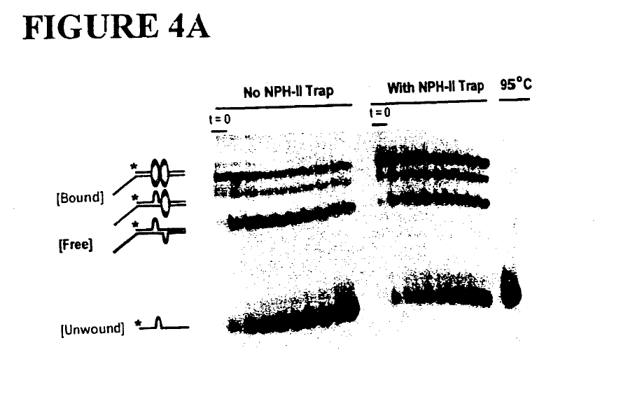

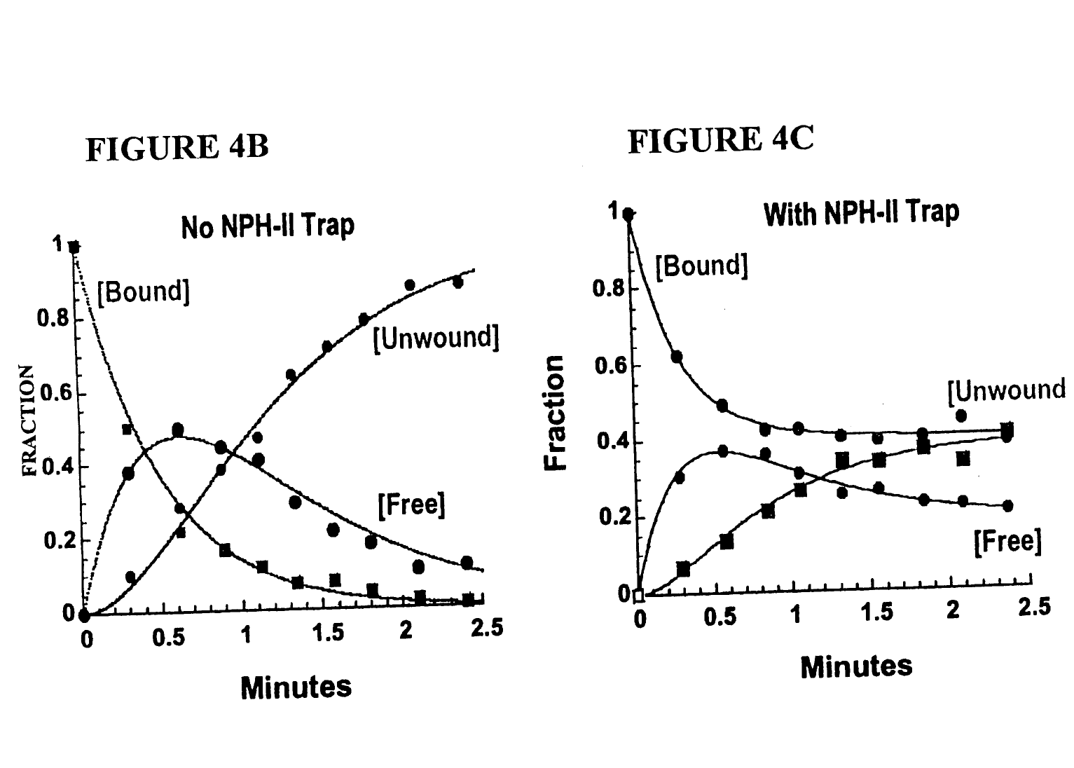

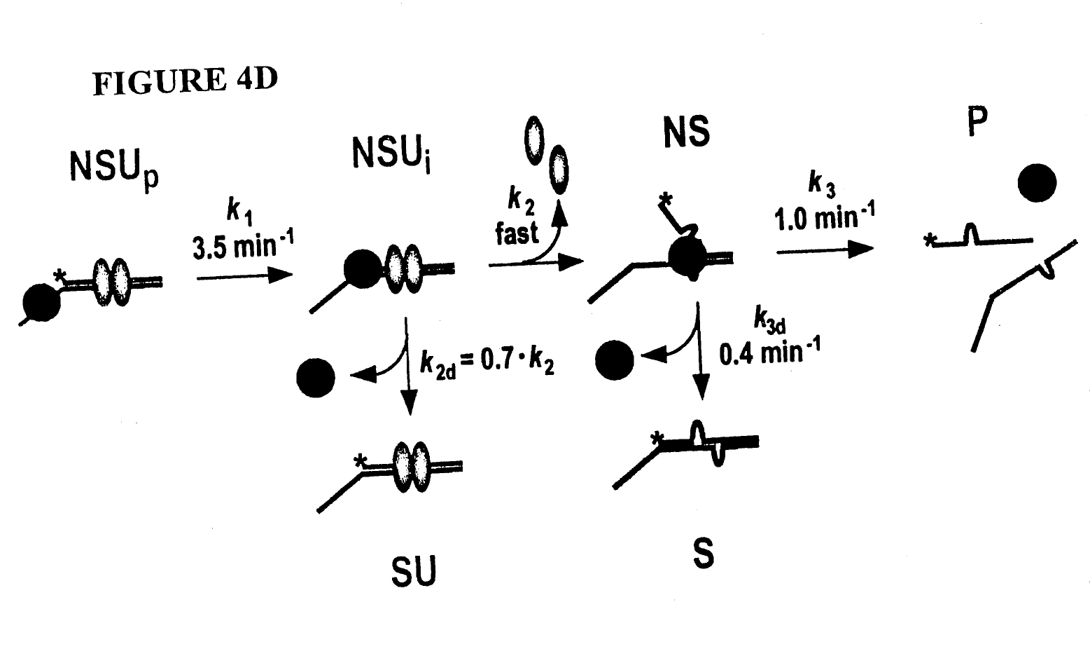

Having established that NPH-II actively displaces U1A in an ATP dependent fashion, it was of interest to determine how U1A binding impedes the helicase activity of NPH-II and to obtain a kinetic framework for the process of protein displacement by a DExH/D protein. To this end, U1A displacement was monitored under single-cycle conditions with respect to NPH-II; that is, any NPH-II that dissociates from the RNA cannot rebind. This was achieved by adding a large excess of trap RNA together with the ATP that is used to initiate unwinding of the NPH-II/substrate/U1A complex (16). In this manner, it was possible to monitor the relative fractions of U1A-bound RNA substrate, free duplex substrate, and unwound RNA strands (FIG. 4A). While the decay of substrate bound to U1A was first order (17), the fraction of free duplex substrate passed through a maximum and the fraction of unwound substrate evolved with a small lag phase (FIG. 4A, C) . This indicates a sequential reaction and suggests the presence of a second slow step after U1A has been displaced.

The most important observation, however, was that a sizeable fraction of the substrate was unwound by NPH-II under single-cycle conditions i.e., NPH-II was able to displace U1A and continue unwinding the substrate without necessarily falling off during the course of reaction. Thus, processivity was not eliminated by the binding of U1A. Nevertheless, U1A caused significant defects in the processivity of NPH-II, as indicated by a plateau in the decay of bound U1A, the fact that the amplitude of free substrate did not return to zero, and that unwinding did not go to completion but only to roughly 40 percent (FIG. 4A, C). Taking all these observations together, it was possible to derive explicit equations describing the timecourses and to model a basic kinetic mechanism for the reaction FIG. 4D, (17)).

In this mechanism, NPH initiates the displacement/unwinding reaction with a rate constant of k₁=3.5 min⁻¹. This rate constant is identical to that of the rate limiting step for unwinding a regular duplex during the NPH-II helicase reaction, which involves a slow step at the junction between the single-strand overhang and duplex region (7). After this initiation step, NPH-II proceeds to displace U1A. This step is fast compared to initiation. The actual rate for U1A displacement is therefore kinetically invisible. However, a lower limit for U1A displacement of k₂>50 min⁻¹ can be estimated (18), which is more then three orders of magnitude faster than the rate of U1A dissociation in the absence of NPH-II and ATP (10⁻² min⁻¹) . Interestingly, even before U1A is displaced, NPH-II dissociates with a rate of 0.7·k2, which explains why only ˜60 percent of U1A molecules are released. After U1A is displaced, another slow step occurs (k₃₌₁ min⁻¹), in which a fraction of NPH-II dissociates from the substrate (k_{3d}=0.4 min⁻¹) . This second slow step (k₃) is strictly dependent on the presence of U1A, and was not observed during unwinding of substrate without U1A (14). It is important to note that the kinetic steps above are likely to describe composite processes, i.e., the rate constants do not necessarily reflect microscopic reaction steps. Analysis of unwinding / displacement under multiple cycle conditions (in which dissociated NPH-II can re-bind the substrate (19), FIG. 4A, left side) indicated that no additional rate altering steps other than re-binding events affect the reaction (FIG. 4B).

Four major mechanistic insights follow from the kinetic analysis: First, physical displacement of U1A is not the slowest step in the reaction, despite the high affinity of U1A to the substrate. Second, NPH-II increases the dissociation rate of U1A by more then three orders of magnitude. Third, NPH-II retains a significant level of processivity while displacing U1A. Fourth, after U1A is displaced, NPH-II needs to be reoriented or repositioned in order to complete substrate unwinding, as suggested by the second slow step (k₃). There are at least two models by which NPH-II accellerates the dissociation of U1A protein: NPH-II may alter the conformation of RNA around the U1A binding site, or it may directly “plow” U1A off the RNA. Although the methods employed here cannot distinguish these scenarios, the presence of intermediate species I2 (FIG. 4D) indicates that U1A displacement does not require the complete unwinding of the RNA duplex, thereby suggesting that a form of “snowplow” model is possible.

By showing that NPH-II actively displaces U1A, this study establishes that DExH/D proteins are capable of efficiently dislodging other proteins from RNA molecules. This ribonucleoprotein displacement, or “RNPase” function, is a new form of enzymatic activity that is driven by ATP hydrolysis and which, like RNA helicase activity, is likely to have many different manifestations in cellular RNA metabolism. The observation that helicase processivity is not eliminated during U1A displacement suggests that DExH/D proteins may be able to switch back and forth between helicase and protein displacement functions, indicating that both activities can reside in the same protein and function in the same macromolecular context (20) . By obviating the need for numerous additional cofactors, this function may considerably simplify the requirements for RNP disassembly or rearrangement during processes such as pre-mRNA splicing or ribosome assembly.

### Part II

The DEXH helicase NPH-II from vaccinia virus (Shuman, 1992) was subjected to quantitative kinetic analysis that varied in composition and length. Kinetics of unwinding were monitored using gel-shift electrophoresis and fluorescence energy transfer methodologies. The RNA substrates contained a single-strand 3′ overhang, which is required for NPH-II unwinding activity (Shuman, 1993). The single-strand 3∝ overhang was identical in length and sequence for every substrate (FIG. 13). Unwinding reactions were preformed under single-turn-over conditions with respect to the RNA substrate. A large excess of nonspecific trap RNA (de la Cruz, 1999) was added to prevent helicase from reassociating with duplex once it falls off during the course of reaction.

In the absence of trap RNA, the unwinding rate and reaction amplitude (defined as the final fraction of unwound RNA) were both insensitive to duplex length (FIG. 13). Each reaction was first-order with a rate constant of 3.5±0.2 min⁻¹ (FIG. 13). In the presence of trap RNA, the rates remained independent of duplex length however, the reaction amplitude decreased with increasing duplex length (FIG. 13). These observations provide three mechanistic insights. First, the fact that long duplexes can be unwound at all in the presence of trap RNA provides strong qualitative evidence that the helicase is a processive enzyme consistent with previous multiple-turnover studies. Second, the dependence of amplitude on duplex length indicates that more protein dissociates from an RNA substrate when the duplex is longer indicating that more protein dissociates from an RNA substrate when the duplex is longer, indicating that more unwinding steps may be required for a long duplex, and there is a specific degree of processivity for the NPH-II enzyme (FIG. 13). Last, the similar rates observed for the unwinding of different duplex lengths indicate that individual unwinding steps do not limit the reaction rate under these conditions. Rate-limiting events during translocation would result in a length-dependent lag phase in each time course.

To evaluate helicase step size and to provide an additional proof of processivity, it was necessary to monitor individual unwinding steps. Conditions were sought under which helicase translocation becomes rate limiting. Variation in temperature and pH did not cause the rates to become increasingly sensitive to duplex length (E. Jankwsky et al., unpublished data); however, marked effects were observed when Mg²⁺, which is the cofactor for ATP hydrolysis, was replaced by Co²⁺. The unwinding reactions displayed pronounced lag phases which increased incrementally with the duplex length (FIG. 13). The data were fit numerically to a kinetic model that relates the translocation rate constant to step size and duplex length (Material and Methods). From this analysis, the unwinding step size of NPH-II was estimated to be six base pairs (bp) and the unwinding rate constant (for each translocative step in Co²⁺at an ATP concentration of 3.5 mM), kᵤ, was 13.4±0.8 min⁻¹. Using the step size of 6 bp, it is possible to calculate a lower limit for the translocation rate constant in Mg⁻². In these conditions, kᵤ was greater than 350 min⁻¹, indicating that translocation in Mg²⁺ is at least 25-fold faster in Co²⁺. The slower unwinding rate in Co²⁺, is not attributable to inhibition of ATPase activity, as kinetic parameters for hydrolysis of ATP are similar in Co²⁺and Mg²⁻ (E. Jankowsky et al., unpublished data).

The existence of a defined step size indicates regularity in the translocation process; that is, unwinding is not random with respect to individual translocation rates and the number of bp displaced in one unwinding step. The step size obtained herein corresponds to about half of a helical turn and is very similar to the unwinding step size of the UvrD DNA helicase of 4-5 bp (Ali and Lohmann, 1997). It is important to note that the unwinding step size is an average value that may not necessarily reflect actual microscopic translocation steps of a helicase (Ali and Lohmann, 1997).

Although the above results suggest that RNA unwinding occurs in consecutive steps, the assays that we used are designed to monitor only the very last event in strand displacement (one does not see a signal until the duplex is completely unwound). Therefore, it is impossible to distinguish between directional translocation and translocation that occurs through random facilitated diffusion (Shimamoto, 1999; Kelemen and Raines, 1999). To determine which of these mechanisms applies, detection of an unwinding intermediate was needed. This was accomplished by using multipiece substrates (MPS), consisting of a continuous bottom strand that is annealed to two adjacent pieces of top-strand RNA (FIG. 7a). The top-strand pieces were generated by site-specific cleavage of a single continuous piece of top-strand RNA (FIG. 15a). The top-strand (first RNA molecule) pieces were generated by site-specific cleavage of a single continuous piece of top-strand RNA using synthetic DNAzyme endonucleases (Santoro and Joyce, 1996). The two resultant pieces had different lengths and could therefore be distinguished by their respective electrophoretic mobilities (FIGS. 15b, 15c). To prevent the results from being biased by the relative length of the top-strand pieces we designed two different sets of MPS (FIG. 15a). All MBPS unwinding reactions were carried out under single-turnover conditions with respect to the RNA substrate, and in the presence of trap RNA.

NPH-II unwound both sets of MPS (FIG. 15b), indicating that the helicase can proceed through nicks in the top strand of the substrate. In the presence of Mg²⁺, all pieces were displaced with apparent first-order kinetics with similar rate constants, k=3.3±0.5 min⁻¹ (FIG. 15b). For both sets of MPS, the piece that was bound farthest from the overhang was displaced with a lower amplitude than the piece located closer to the single-strand 3′ overhang (FIG. 15b). This implies that fewer unwinding steps were required for the displacement of the piece closer to the overhang. The fact that these effects depend exclusively on the location of the top strands relative to the single-strand 3′ overhang (rather than their length) strongly suggests that the strand displacement progresses in a distinct 3′-to-5′ direction with respect to the bottom strand.

The similar unwinding rates for both pieces of top-strand RNA indicate that neither a late step in the reaction, such as the final strand separation, nor translocation itself, limits the rate of the overall process in Mg²⁺. A late rate-limiting step would result in a slower rate for unwinding of the piece farthest from the overhang because two strand separations are required for unwinding of the second piece. A rate-limiting translocation would cause a lag phase in the time courses as observed in the presence of Co²⁺ (FIG. 14). Thus in the presence of Mg²⁺,an initiation step at the very begining of the unwinding process limits the rate of the overall reaction.

By contrast, reactions conducted in the presence of Co²⁺ displayed a pronounced lag phase attributable to rate-limiting translocation steps (FIG. 15c). Notably, the displacement of the two pieces occurred with different lag phases. The lag was largest for the piece farthest from the overhang, reflecting an increase in the number of unwinding steps that are required as the helicase moves away from the single-strand 3′ overhang. The effects were exclusively dependent on the location of the pieces relative to the over hang and not on their length indicating a distinct 3′-to-5′ directionality in the progression of unwinding with respect to the bottom strand.

Having established that the helicase unwinds RNA substrates in a processive directional fashion with a distinct unwinding step size it was of interest to correlate this process with the role of ATP. The effect of ATP on enzyme processivity (P) was of particular interest (FIG. 16). Processivity reflects the probability that the helicase will perform the next unwinding step rather than dissociate from the substrate (Lohmann and Bjoernson, 1996). Quantitation of processivity was carried out by determining the reaction amplitude in the presence of trap RNA, such that all dissociation of protein from the RNA substrate during unwinding was effectively irreversible. Processivity decreased with decreasing ATP concentration (FIG. 16a), which indicates that the helicase dissociates more readily from RNA in non-ATP-bound states than in the ATP-bound state, consistent with previous studies of RNA binding by NPH-II (Shuman, 1992). In addition, the processivity at ATP saturation approaches P=1, regardless of whether Co²⁺ or Mg²⁺ are the cofactors for ATP hydrolysis (FIG. 16a). This indicates that NPH-II in the ATP-bound state does not significantly dissociate from the RNA substrate, and that dissociation of protein from RNA occurs predominantly in non-ATP-bound states. This is highly instructive because it shows that ATP binding and not just the consumption of ATP through hydrolysis, is of critical importance in the helicase mechanism.

Although RNA unwinding activity by NPH-II is dependent on ATP hydrolysis (Shuman, 1992), one must establish quantitative link between ATP utilization and a specific amount of unwinding to conclude that DExH/D proteins behave as true molecular motors (Schnapp, 1995). Plots of reaction amplitude versus ATP concentration resulted in sigmoidal curves (FIGS. 16b, 16c) that are affected by duplex length: the longer the duplex, the more pronounced the sigmoidal shape of the time course. These results, together with the observation of a discrete step size and the fact that NPH-II does not dissociate in the ATP-bound state are consistent with the kinetic scheme shown in FIG. 17. This scheme was used to derive an explicit equation that describes the plots of reaction amplitudes versus ATP concentration (FIGS. 16b, 16c).

$$A = ( 1 - x ) \cdot {( 1 + \frac{{k}_{c}}{{k}_{U [ S ]}} \cdot \frac{{K}_{d}^{′}}{[ ATP ]} )}^{- υ m}$$

The fit of this expression to the data establishes a direct relationship between processivity and ATP concentration, indicating that translocation is coupled to the binding of ATP. This link between ATP utilization and directional movement supports the assertation that NPH-II is a processive molecular motor. The results suggest the following basic mechanism for RNA duplex unwinding by the DExH RNA helicase NPH-II (FIG. 17); unwinding is preceded by a slow initiation step (2.3±0.2 in Co²⁺, 3.5±0.3 in Mg²⁺). Subsequent unwinding occurs in distinct consecutive steps of roughly one-half helix turn in a defined 3′-to-5′ direction with respect to the single-strand 3′ overhang. The helicase translocates rapidly along the RNA substrate, unwinding RNA with a rate that is dependent on identity of the metal ion cofactor (13.4±0.8 min⁻¹ per step for Co²⁺; ≧350 min-⁻¹ per step for Mg²⁺) . During each step, a fraction of protein, predominantly in non-ATP-bound states, dissociates from the RNA. The dependence of reaction on ATP hydrolysis and the direct connection between ATP binding and translocation indicate that NPH-II is a processive directional motor for unwinding RNA. This activity DexH/D proteins might ensure processive, regulated rearrangement of structured RNA in macromolecular assemblies during processes such as RNA splicing, export or translation initiation. A rate-limiting step before translocation would provide a straight-forward way to control unwinding by blocking and deblocking an early initiation event. Alternatively, factors that control the local concentration of ATP or accessiblity of the ATP binding site on the protein could regulate processivity, enabling the helicase to dissociate from RNA at appropriate times.

DExH/DEAD box proteins (putative RNA helicases) are important in all aspects of RNA transcription, maturation and translation. A step towards understanding the function(s) of this class of enzymes at the molecular level is to investigate the mechanism underlying duplex RNA unwinding by the vaccinia virus DEXH RNA helicase NPH-II.

Transient kinetic experiments were performed using a series of fluorescent dye labeled RNA substrates with duplex regions of different length. The unwinding reaction was monitored continuously in real time by measuring the changes in fluorescence energy transfer upon the unwinding reaction.

Timecourses under single turnover conditions displayed significant dependence on the length of the duplex region in terms of reaction rate and reaction amplitude. Reaction rates were also affected by the nature of the divalent cation cofactor. In the presence of Co²⁺ timecourses were found to have a significant lag phase, increasing with the duplex length. In the presence of Mg²⁺ no comparable lag phase was observed, but reaction rate as well as reaction amplitude were greater as compared to the reactions with Co²⁺. Dependent on the duplex length, the ATP concentration affected the reaction rate and amplitude with both metal cofactors.

These findings suggest a basic mechanism for the translocation/unwinding reaction consisting of consecutive events of ATP binding and translocation. Quantitative description of this mechanism leading to the rate and step size for the translocation/unwinding reaction follows.

Design of the RNA substrates. These studies used the RNA helicase, vaccinia virus DExH protein NPH-II. The protein was expressed in baculavirus infected insect cells (Gross and Shuman,1995) The NPH-II helicase requires the substrate to have single stranded overhangs 3′-end to the duplex region (Shuman, 1992), which are necessary for the helicase to bind the substrate (Shuman, 1993). In order to prevent binding of the protein at multiple sites, a substrate for mechanistical studies should contain solely one single stranded region. This single stranded region should be constant for a series of substrates where the length of the duplex region is variable.

Two different ways to design the substrates: by chemical synthesis with subsequent template directed ligation (Moore and Sharp, 1992) and by in vitro transcription from PCR generated templates (Chabot, 1992) with subsequent DNAzyme processing (Santoro and Joyce, 1997) in order to produce perfect blunt ended duplexes. Chemical synthesis bears the advantage of complete flexibility in the sequence choice and allows the incorporation of a variety of modifications including nucleoside and backbone modifications as well as fluorescent labels. The method is limited in terms of the length of the oligos which can be produced. In vitro transcription from PCR generated templates does not have these limitations for the length of the oligos but this method is not completely variable concerning the sequence and it does not allow the incorporation of extensive modifications.

Exploiting the respective advantages of both methods we designed two series of substrates. The series based on chemical synthesis contains a U-35 single stranded overhang and duplex regions from 12 to 36 basepairs (FIG. 6). The series based on in vitro transcription contains a 33 mucleotide overhang and duplex regions from 36 83 basepairs. The unwinding reaction of the substrates was monitored by separation of unwound strands from intact substrate on native gel electrophoresis. All substrates tested were unwound by NPH-II (FIG. 6).

Effect of trap RNA on the unwinding reaction. Excess single stranded RNA (Trap RNA) is required for single turnover reactions where dissociation of protein from the substrate has to be irreversible, i.e. trap RNA has to prevent re-binding of protein to the substrate during the reaction (FIG. 7a). The effect of trap RNA addition with two substrates was tested: one with a 36 bp duplex region and one with a 83 bp duplex region. Reaction rates and amplitudes without trap addition were comparable with both substrates (FIG. 7b). The addition of trap RNA did not significantly change the reaction rates with both substrates. However, reaction amplitudes decreased with both of the substrates as compared to the reactions without trap (FIG. 7b), whereby the reaction amplitude with the longer substrate (LS83) was lower than the amplitude with the shorter substrate (LS36). Thus, the duplex length does not affect the rate of unwinding suggesting that the rate-limiting step for the overall reaction is an event which is not a potential translocation. However, the different reaction amplitudes indicate that protein dissociates off the substrate more frequently during the of unwinding of longer duplexes, suggesting that the number of dissociation “events” increases with increasing duplex length. This is an indirect indication that there are distinct consecutive steps in the unwinding reaction, even if this is not reflected in the reaction rate (FIG. 5)

Monitoring duplex unwinding by fluorescence measurements. In order to obtain data of higher time resolution, the unwinding reaction was monitored by measuring changes in fluorescence in real time upon the unwinding of fluorescently labeled duplexes (FIG. 8a). Since the data obtained by gel shift and by fluorescence are superimposable, the real time fluorescence method provided also the control that no artifacts were introduced by the gel separation method (FIG. 8b). The reaction rates with both substrates tested (18 bp duplex and 36 bp duplex) were similar (FIG. 8b). The reaction amplitude of the 36 bp duplex was lower than that of the 18 bp duplex. Thus, these observations agree with the data described above (FIG. 7)

Effect of the ATP concentration on the unwinding reaction. The decreasing reaction amplitude with increasing duplex length in reactions where protein dissociation from the substrate was irreversible (FIGS. 7 and 8) indicates that with each “unwinding step” a certain fraction of protein dissociates from the substrate (FIG. 5). For the understanding of the mechanism of unwinding it is of importance to know whether this dissociation occurs in an ATP-bound state of the protein or in a non-ATP-bound state or in both states. Experimentally, this question can be accessed by measuring the effect of ATP concentration variation on the reaction amplitude. Decreasing reaction amplitudes with decreasing ATP concentrations would indicate that dissociation occurs in a non-ATP-bound state. Dissociation in an ATP-bound state would be indicated by differences in the reaction amplitudes with duplexes of different length under ATP saturation of the system. Measurements conducted of the reaction amplitude in dependence on the ATP concentration with a 36 bp substrate and a 83 bp substrate (FIG. 9). The reaction amplitude decreased with decreasing ATP concentrations, indicating clearly that dissociation occurs at non-ATP bound states of the protein. The differences in the reaction amplitude of both substrates near ATP saturation suggest further that the protein dissociates also in the ATP-bound state. (FIG. 9).

Effect of Mg²⁺ substitution on the unwinding reaction. At the reaction conditions tested so far, the only response to the variation in the duplex length was the reaction amplitude. Although this suggests a processive unwinding reaction, the fact that no effect was found on the rates of the unwinding reaction indicates that not a potential translocation limits the overall rate of the reaction. However, for the quantification of the unwinding steps in terms of the step size and individual rate constants it is necessary that the translocation directly affects the reaction rate. Therefore, attempts were made to decrease the rate of translocation relative to the rate limiting step such that intermediates of the unwinding reaction would be kinetically detectable and translocation would be directly reflected in the unwinding timecourse (FIG. 5). Variation in pH and temperature did not result in the desired changes in the timecourses (not shown). Subsequently, we examined whether a substitution of Mg²⁺ with other divalent cations, which act as cofactor for the ATP hydrolysis, provides reaction conditions where the translocation would be slower relative to the previously rate limiting step. Recently, only Mn²⁺ and Co²⁺ were found to substitute for Mg²⁺ in the unwinding reaction for NPH-II (Shuman, 1992). In reactions with RNA-protein pre-annealing and in the presence of trap RNA, Mn²⁺ did not change the timecourses as compared to the reactions with Mg²⁺ (FIG. 10). However, substituting Mg²⁺ with Co²⁺ resulted in a substantial change in the observed timecourse (FIG. 10). With Co²⁺, the timecourse displayed a pronounced lag phase, suggesting reaction intermediates (FIG. 5). This assumption was supported by measuring reactions with substrates with duplex regions of different length in the presence of Co²⁺. The timecourses showed an increasing lag phase with increasing duplex length, i.e. the longer the duplex, the more intermediates appear in the unwinding reaction (FIG. 11). This indicated that in the presence of Co²⁺, it can be directly observed that the reaction proceeds in distinct consecutive steps and can therefore be considered to be processive. In accordance with the results described above (FIGS. 6 and 7), the reaction amplitude decreased with increasing duplex length (FIG. 11), indicating that protein dissociates more frequently during the unwinding of longer duplexes.

Employing multi-piece substrates for probing the directionality of the unwinding reaction. The results described above (FIGS. 6-11) suggested that the unwinding of RNA duplexes by NPH-II is in fact processive. The second basic feature of the unwinding reaction, the directionality of the process, could not be accessed yet. Since monitoring the unwinding reaction with the described methods bears the disadvantage that the observed unwinding is caused by an event at the end of the unwinding reaction (FIG. 5), no direct evidence for directionality can be provided. Although a processive unwinding together with the binding of the protein at the single stranded overhang of the substrate indirectly argues for a certain directionality of the process, a number of alternative unwinding mechanisms (for instance looping of the single-stranded-bound protein into the duplex region) cannot be ruled out. In order to prove directionality unambiguously, we attempted to employ a more direct assay, where, beside, the unwinding of the “whole” duplex at least one intermediate has to be monitored during the reaction. We accomplished that by designing multi-piece-substrates (MPS), which comprise a regular bottom strand but two adjacent to each other binding top strand pieces (FIG. 12a). Provided that the two pieces can be separated from each other on native PAGE, it should be possible to observe the separate displacement of the two pieces as the helicase translocates in a directional way. In order to exclude effects of the length of the different pieces per se, we designed two MPS; one with the longer piece next to the single stranded overhang, and another one with the shorter piece next to the single stranded overhang (FIG. 12a). The unwinding reactions were performed with protein-RNA pre-annealing and in the presence of trap RNA, such that dissociation of protein from the substrate was irreversible.

Both MPS were unwound by NPH-II, emphasizing that the helicase tolerates nicks in the top strand of the substrate. In the presence of Mg²⁺, both pieces were displaced with a similar rate (FIG. 12b). The piece binding more distal to the overhang was displaced with a lower amplitude as compared to the piece binding closer to the single stranded overhang. This was observed for both MPS, and, therefore, the amplitude effects are caused only by the position of the pieces relative to the overhang. These results support the findings with the regular substrates (FIGS. 6, 7), where, although more protein dissociates from the substrate with increasing duplex length, the reaction rate is not limited by the translocation. Moreover, the fact that the amplitude was lower for the piece more distal to the overhang but the reaction rates of both pieces were similar suggests that the event which limits the rate of the overall reaction is before the translocation process, i.e. the unwinding initiation is likely to limit the overall reaction in the presence of Mg²⁺.

In the presence of Co²⁺, the displacement of both pieces occurred with different lag phases (FIG. 12c). The lag for the pieces next to the overhang was always smaller, than the lag for the pieces more distal to the overhang (FIG. 12c), indicating that one piece is displaced after the other and that this displacement process has a directionality: initiating from the single stranded region the helicase translocates towards the other end of the duplex while it unwinds it. Moreover, the reaction amplitude for the piece more distal to the overhang was smaller then the amplitude for the piece next to the overhang, indicating that also in the presence of Co²⁺ protein dissociates from the substrate more frequently with increasing duplex length.

Materials and Methods

NPH-II was expressed and purified as described (Gross and Shuman, 1995). RNAs were prepared by in vitro transcription of PCR-generated T7 transcription templates (Jankowsky and Schwenzer, 1996) or by chemical synthesis (Wincott et al., 1995). The PCR-amplified DNA templates represent a segment of the ampicillin-resistance gene of the pBS(+/−) phagemid (Stratagene). To generate perfect blunt-end duplexes, the RNA pieces were trimmed by site-directed processing using engineered DNAzymes (Santoro and Joyce, 1997; Pyle et al., 2000) with 12 nucleotides on each of the binding arms. Bottom-strand RNAs were joined from two separately synthesized oligonucleotides by template-directed ligation using T4 DNA ligase (Moore and Sharp, 1992) RNA duplexes were formed by combining the botton-strand RNA with a five-fold molar excess of γ³² P-labelled top strand in 10 mM MOPS, 6.5, 1 mM EDTA. The solution was heated to 95° C. for 2 min and cooled to room temperature over 90 min. Duplexes were separated from single-strand RNA by native PAGE, visualized by radiolytic scanning (Packard Instant Imager) and excised from the gel.

Unwinding Reactions

Unwinding reactions were performed at room temperature in 30 μl of 40 mM Tris-HCl buffer, pH 8.0 and 4 mM MgCl₂ or CoCl₂. The reaction also contained 25 mM NaCl, which was introduced with the protein storage buffer. In a typical reaction, 1-2 nM RNA substrate was incubated with 10-15 nM NPH-II in reaction buffer without ATP at room temperature for 5-7 min. Longer pre-incubation time did not change the reaction kinetics. Saturation of substrate with protein before the reaction was verified by gel-shift analysis (Lohmann and Bjoernson, 1996). The unwinding reaction initiated by adding the ATP to a final concentration of 3.5 mM unless otherwise stated. Aliquots at the respective time points were quenched with two volumes of stop buffer 25 mM EDTA, 0.4% SDS, 0.05% BPB, 0.05% XCB, 10% glycerol containing 200 nM of unlabelled top-strand RNA to prevent re-annealing of unwound duplexes during electrophoresis. Reannealing of unwound duplexes during the reaction did not affect unwinding kinetics under any reaction conditions as verified by independent measurements of association rate constants of the substrate strands- Excess RNA (Trap RNA) (200 nM final concentration added with the ATP) was a single-strand of 39-nucleotides of unrelated sequence. Higher concentrations of trap RNA did not change observed reaction amplitudes.

Unwinding reactions were also monitored in real time using fluorescence energy transfer experiments on substrates labelled at the 3′ and 5′ ends of the duplex terminus farthest from the single stranded 3′ overhang. These measurements were superimposable with results from gel-shift measurements and confirmed the first-order nature of the time courses as well as the rate constants.

Kinetic Analysis

Data were fit numerically using the FITSIM software package (Barshop and Frieden, 1983). Time courses were corrected for the reaction amplitudes, and initial kinetic parameters were determined using the KINSIM software (Barshop and Frieden, 1983). Unwinding step size and rate constants were determined from time courses in Co²⁺ using a kinetic model in which the unwinding reaction consists of N consecutive first-order reactions where N is the number of kinetic steps. Rate constants (except for the first step see below) were linked, that is, forced to yield the same value in the fitting. The linked rate constants and the first rate constant were allowed to float simultaneously. On the basis of the observation than an initial step is slower than subsequent steps in Mg²⁺ (see FIG. 15b and text), the first kinetic step was defined as having a different rate constant (k₁)than subsequent kinetic steps (k^{u}) Using this model, increasing numbers of steps (N) were examined iteratively for each duplex until k₁ and kᵤ approached constant values for the different duplexes. Convergence occurred when k₁=2.3±0.2 min⁻¹ and kᵤ=13.4±0.8 min⁻¹.

Note that kᵤ is an apparent rate constant that depends on the ATP concentration according to kᵤ=k_{u[s]}[ATP/(ATP+K′_{d}) , where k_{u[S]} is the respective rate constant at ATP saturation. The step size (m) was calculated according to m=L(N−y), where y is the number of kinetic steps do not contribute to the actual strand displacement and L is the duplex length. A step size of m=6 and a value of y=1 were consistently calculated for all duplexes λ including multi-piece substrates.

The lower limit for the translocation rate constant in Mg²⁺ was estimated by considering that a lag phase was not observed with the longest (83 bp) duplex (FIG. 13b). Assuming an unwinding step size identical to that in Co²⁺ (6 bp), and that kᵤ must be equal or greater than a value that does not cause an apparent lag phase in 14 unwinding steps, the translocation rate constant was estimated as kᵤ>˜350 min⁻¹.

Equation (1) was derived from the expression describing the dependence of reaction amplitude (A) on the processivity (P):

A=(1−x)P

[symbol]

where X is the fraction of protein that dissociates from the substrate before unwinding occurs. P is then substituted with a term that relates processivity and ATP concentration under the conditions specified by the scheme in FIG. 17.

$$P = \frac{{k}_{insp} ( \frac{\vert ATP \vert}{{K}_{d}^{′} + \vert ATP \vert} )}{{k}_{LTST} ( \frac{[ ATP ]}{{K}_{d}^{′} + [ ATP ]} ) + {k}_{p} ( 1 - \frac{[ ATP ]}{{K}_{d}^{′} + [ ATP ]} )}$$

where K′_{d} is the apparent dissociation constant of ATP from the enzyme substrate complex.

RNA Substrate Preparation

Chemical Synthesis:

RNA oligonucleotides were prepared by chemical synthesis using phosphoramidite chemistry (amidites purchased from Glen Research) on an ABI 392 RNA/DNA synthesizer. Deprotection of the crude oligonucleotides was carried out according to standard protocols (Wincott et al., 1995). All RNA oligonucleotides were purified on denaturing PAGE.

The bottom strand RNAs were joined out of two separately synthesized oligonucleotides by template directed ligation using T4 DNA ligase (Moore and Sharp, 1992). The ligated products were purified on denaturing PAGE.

Fluorescent Labeling:

Amino linkers were placed in the 3′ or 5′ position of the respective RNA during the synthesis. After removal of all deprotection groups, the modified RNA was labeled with fluorescein-or rhodamine-isothiocyanate (Sigma), respectively. Labeling reactions were carried out in the dark for 12-18 hours with 50 mM dye-isothiocyanate and RNA concentrations below 50 μM in 0.1 M sodiumbicarbonate (pH 9.0) and 20% (v/v) DMSO for the fluorescein-isothiocyanate labeling and 50% (v/v) DMSO for the rhodamine-isothiocyanate labeling. The excess rhodamine-isothiocyanate was removed by repeated phenol extractions followed by ethanol precipitation. The excess fluorescein-isothiocyanate was removed by repeated ethanol precipitations. All labeled RNAs were finally purified on denaturing PAGE.

In vitro transcription: RNAs for the multi-piece-substrates were prepared by in vitro transcription of PCR generated templates (Chabot, 1992). The PCR-amplified DNA-templates represent a segment of the ampicillin resistance gene of the pBS (+/−) phageimid (Stratagene). Templates for bottom and top strand RNAs were separately amplified from ScaI linearized phageimid using different pairs of primers with the respective sense primer containing the binding sequence as well as the T7 promoter sequence. 100 μL PCR reactions were carried out for 30 cycles (52° C., 72° C., 94° C., 1 min each) followed by a 7 min final extension at 72° C. Homogeneity of the PCR products was verified on a 2% agarose gel. The PCR reaction solutions were extracted with an equal volume of phenol, followed by extraction with chloroform isoamylalcohol (Sambrook et al., 1989). Not incorporated dNTPs were removed by SEC (NAP-5, Pharmacia). Subsequently, the volume of the solution was reduced in a Speedvac concentrator (Savant). The obtained DNA templates were used for in vitro transcriptions with T7 polymerase according to standard procedures. The transcribed RNAs were purified on denaturing PAGE.

The top strands for the Multi-Piece-Substrates were produced by site directed processing using an engineered DNAzyme (Santoro and Joyce, 1997). DNAzymes with 12 nucleotides in each of the binding arms were designed to cut at the desired positions. The DNAzyme reactions (10 μM RNA, 30 μM DNAzyme) were carried out for 4 h at 40° C. followed by ethanol precipitation. Subsequently, the DNAzyme was destroyed by RQ RNAse free DNAse (Promega) in a 100 μL volume with 15 units DNAse for 60 min at 37° C. The RNA was ethanol precipitated and the products were purified by denaturing PAGE.

Duplexes were formed by combining the bottom strand RNA with a 5 fold molar excess of labeled (either radioactively or fluorescently) top strand in 10 mM MOPS (pH 6.5), 1 mM EDTA. The solution was heated to 95° C. and cooled to room temperature within 90 min. Duplexes were separated from single stranded RNA on native PAGE. The RNA was visualized either by UV-shadowing or by radiolytic scanning (Packard Instant Imager), cut out, eluted, and ethanol precipitated.

Helicase Reactions

Unwinding reactions were carried out at room temperature in 40 mM Tris/HCl (pH 8.0) and 3 mM MgCl₂ (MnCl₂, CoCl₂), 20 mM NaCl was present in the reaction due to introduction with the protein storage buffer. In a typical reaction, 3 nM RNA substrate was incubated with 10-15 nM NPH-II in reaction buffer without ATP at room temperature for 7 min. Longer incubation time did not change the observed reaction kinetics. The reaction was started by adding the ATP. In reactions with trap RNA, the trap was added together with the ATP at the reaction start.

Gel Shift Measurements:

For the reactions monitored by gel shift PAGE, reactions were performed in a volume of 30 μL. Aliquotes were taken at appropriate times and combined with two volume of stop buffer (25 mM EDTA, 0.4% SDS, 0.05-BPB, 0.05% XCB, 10% glycerol), containing 200 nM of unlabeled top strand in order to prevent re-annealing of unwound duplexes during electrophoresis. Unwound strands were separated from intact substrate on native PAGE and the amount of unwound duplex was determined using a Molecular Dynamics PhosphorImager and the Imagequant Software.

Fluorescence Measurements:

For the reactions monitored by fluorescence measurements, reactions were performed in fluorescence cuvette (600 μL) in an Aminco SLM AB2 fluorescence spectrometer. Excitation wavelength was set at 492 nm, (8 nm slid width) emission was recorded at 518 nm (8 nm slid width, PMT sensitivity 600-800V). The reaction was started as described and measurements were taken automatically in fixed intervals. Employing a series of designed model substrates, it was shown that the unwinding of RNA duplexes by the DEXH protein NPH-II is processive. In the presence of Mg²⁺ the translocation does not limit the overall reaction rate. The rate-limiting step of the unwinding reaction is likely to be the unwinding initiation. The sensitivity of the reaction amplitude to the duplex length indicates that during each “unwinding step” a certain fraction protein dissociates from the substrate. The dependence of the reaction amplitude on the ATP concentration emphasizes that this dissociation occurs in non-ATP bound states of the protein as well as in ATP-bound states of the protein.

Also developed was an assay to test the directionality of the unwinding process and showed that the unwinding process has a defined directionality: initiating from the single stranded region the helicase translocates towards the other end of the duplex while it unwinds it. The proof of the processivity and directionality of the unwinding process is the prerequisite for quantitative characterization of the helicase reaction which is necessary to derive functional models.

References

1. J. de la Cruz, D. Kressler, P. Linder, TIBS 24, 192 (1999).

2. J. P. Staley and C. Guthrie, Cell 90, 1041 (1998).

3. G. Kadare and A. L. Haenni, J. Virol. 71, 2583 (1997).

4. B. Schwer and C. Guthrie, EMBO J. 11, 5033 (1992).

5. P. L. Raghunathan and C. Guthrie, Curr. Biol. 8, 847 (1998).

6. J. D. O. Wagner, E. Jankowsky, M. Company, A. M. Pyle, J. N. Abelson, EMBO J. 17, 2926 (1998).

7. E. Jankowsky, C. H. Gross, S. Shuman, A. M. Pyle, Nature 403, 447 (2000).

8. U1A containing residues 1-117 was expressed and purified as described (24). NPH-II was expressed in cultured insect cells infected with recombinant baculovirus and purified as described (25). Purity of both proteins (>95%) was assessed by SDS PAGE and staining of the polypeptides with Coomassie brilliant blue.

9. R. J. Grainger, D. G. Norman, D. M. Lilley, J. Mol. Biol. 288, 585 (1999).

10. D. Scherly, W. Boelens, N. A. Dathan, W. J. van Venrooij, I. W. Mattaj, Nature 345, 502 (1990).

11. G. Varani and K. Nagai, Annu. Rev. Biophys. Biomol. Struct. 27, 407 (1998).

12. L. Varani et al., Nature Struct. Biol. 7, 329 (2000).

13. To unwind RNA, NPH-II requires a single strand overhang 3′ to the duplex region (26). Optimal helicase activity requires a 3′ overhang of at least 20 nucleotides (27). The affinity of NPH-II for blunt-end duplex RNA is low: no significant binding is observed at nanomolar concentrations of NPH-II (27).

14. Unwinding of substrate in the absence of U1A can be described by a single exponential with a rate constant of k_{unwinding}=3.5±0.4 min⁻¹, which is in agreement with rate constants measured for unwinding of regular duplexes under these conditions (7). In the presence of NPH-II trap, ˜70% percent of substrate was unwound, i.e., the overall processivity for unwinding this substrate is slightly lower then the overall processivity for unwinding a regular duplex with the same number of basepairs (7). It should be noted that U1A did not affect unwinding reactions with regular duplexes at the concentrations used (27).

15. The RNA used for trapping dissociated U1A was based on the hairpin that forms the U1A binding site in the U1 snRNA (10) An RNA oligonucleotide of the sequence 5′-GGAGAACCAUUGCACUCCGGUUCUUC was prepared by chemical synthesis and purified as described (23)

16. NPH-II trap consisted of a 12 basepair duplex with 24 nucleotide single strand overhang which was formed out of two strands with the sequence: 3′ ACGAGGGAGACGAGGAGACGGAGCGACGGCAGCGGU and 5′ CUGCCGUCGCCA. RNAs were synthesized and purified, and the duplex was formed as described (23, 7).

17. Explicit equations describing the kinetic mechanism (FIG. 4D) were derived by considering the species NSUᵢ as a fast intermediate such that d[NSU]i/dt=0. The relative fractions of bound, free and unwound substrate were described by:

$$\begin{matrix}\begin{matrix}\begin{matrix}\begin{matrix}frac [ bound ] = \frac{{k}_{2 d}}{{k}_{2} + {k}_{2 d}} \cdot ( 1 - {ⅇ}^{- {k}_{1} t} ) + {ⅇ}^{- {k}_{1} t} \\ frac [ free ] = \frac{{k}_{2}}{{k}_{2} + {k}_{2 d}} \cdot [ \frac{{k}_{1}}{{k}_{3} + {k}_{3 d} - {k}_{1}} \cdot \frac{{k}_{3}}{{k}_{3} + {k}_{3 d}} \cdot \end{matrix} \\ ( {ⅇ}^{- {k}_{1} t} - {ⅇ}^{- ( {k}_{3} + {k}_{3 d} ) t} ) + \frac{{k}_{3 d}}{{k}_{3} + {k}_{3 d}} ( 1 - {ⅇ}^{- {k}_{1} t} ) ]\end{matrix} \\ frac [ unwound ] = \frac{{k}_{2}}{{k}_{2} + {k}_{2 d}} \cdot \frac{{k}_{3}}{{k}_{3} + {k}_{3 d}} \cdot \end{matrix} \\ [ 1 - {ⅇ}^{- {k}_{1} t} - \frac{{k}_{1}}{{k}_{3} + {k}_{3 d} - {k}_{1}} \cdot ( {ⅇ}^{- {k}_{1} t} - {ⅇ}^{- ( {k}_{3} + {k}_{3 d} ) t} ]\end{matrix}$$

These equations were used to fit the normalized (22) time courses of reactions conducted in the presence of NPH-II trap RNA. Fitting was performed using Kaleidagraph (Synergy software). Values for k₂/(k₂+k_{2d}) and for k₁ were obtained by fitting the timecourse of fraction[Bound]. Values for k₃ and k_{3d} were computed by fitting fraction[Free] and fraction[Unwound] with fixed k₂/k₂d and k₁. The rate constants provided are average values calculated from three different time courses, resulting in: k₁=3.52±0.15 min⁻¹, k₂/(k₂+k_{2d})=0.59±0.02, k₃=1.04±0.04 min⁻¹, k_{3d}=0.40±0.12 min⁻¹.

18. The lower limit for k₂ was estimated by simulating the timecourse using the empirically-determined rate constants, but decreasing the values for k₂. Noticable deviation from the observed timecourse was detected for values of k₂<50 min⁻¹, i.e., the actual constant k₂ is necessarily larger than this value.

19. For simulating the reaction without NPH-II trap, re-binding of helicase to substrate was considered by adding three steps to the reaction scheme in FIG. 4D: (i) Fast binding of helicase to substrate-U1A complex: N+SU→NSUₚ, where k₆=10⁹ mol⁻¹·min⁻¹ and the initial NPH-II concentration N₀=20 nM. (ii) Fast re-binding of helicase to substrate, without U1A bound: N+S→NS_{d}, where k₇=10⁹ mol⁻¹·min⁻¹ and the initial NPH-II concentration N₀=20 nm. (iii) Unwinding of re-bound substrate without U1A bound: NS_{d}→P, where k₈=3.5 min⁻¹ (12). Note that step (iii) represents multiple reactions. Simulations were performed with normalized (22) time courses using the KINSIM software package (28).

20. This contrasts with the SNF2 family protein Mot1p, which displaces the TATA- box binding protein from DNA in an ATP-dependent fashion (30), but which lacks helicase activity.

21. C. W. van Gelder et al., EMBO J. 12, 5191 (1993).

22. Amplitudes were corrected for the final reaction endpoint (normalized). Endpoints (t→∞) were determined after 10 minutes of reaction without NPH-II trap. The endpoint values were determined to be: frac[Bound]_{obs}(t→∞)=0.04, frac[Free]_{obs}(t→∞)=0.02, frac[Unwound]_{obs}(t→∞)=0.94. Amplitudes at a given time, t, were corrected as: frac[Bound](t)=(frac[Bound]_{obs}(t)−0.04)/(1−0.04), frac[Unwound](t)=frac[Unwound]_{obs}(t)/0.94, frac[Free](t)=1-frac[Bound](t)−frac[Unwound](t).

23. RNA oligonucleotides were prepared by chemical synthesis on an ABI 392 RNA/DNA synsthesizer using phosphoramidite chemistry (Reagents purchased from Glen Research). Crude oligonucleotides were deprotected according to standard protocols (28) and purified by denaturing PAGE. Duplexes were formed and purified as described previously (7).

24. J. M. Avis et al., J. Mol. Biol. 257, 398 (1996).

25. C. H. Gross and S. Shuman, J. Virol. 69, 4727 (1995).

26. S. Shuman, J. Biol. Chem. 268, 1179B (1993).

27. E. Jankowsky et al., unpublished results.

28. B. A. Barshop and C. Frieden, Anal. Biochem. 130, 134 (1983).

29. Wincott et al., Nucleic Acids Res. 23, 2677 (1995).

30. D. T. Aubele, D. Wang, K. W. Post, S. Hahn, Mol. Cell Biol. 17, 4842 (1997).

Ali, J. A., and Lohmann, T. M. (1997) “Kinetic Measurement of the Step Size DNA Unwinding by Escherischia coli UvrD Helicase”, Science, 275, 377-380.

Barshop, B. A. and Frieden, C. (1983) “Analysis of Numerical Methods For Computer Simulation of Kinetic Processes: Development of KINSIM—A Flexible Portable System. Anal. Biochem. 130, 134-145.

Chabot, B. (1992) “Synthesis and Purification of RNA Substrates, in RNA Processing: A Practical Approach”, Higgins, S. J. and Hames, B. D. Eds., IRL Press, Oxford, U.K., pp 1-31.

de la Cruz, I., Kresler, D. and Linder P. (1999) “Unwinding RNA in Saccharomyces Cerevisiae: DEAD-Box Proteins and Related Families,” Trends Biochem. Sc. 24, 192-198.

Gross, C. H. and Shuman, S. (1995) “Mutational Analysis of Vaccinia Virus Nucleoside Triphosphate Hydrolase II, a DExH Box RNA Helicase”, J. Virol., 69, 4727-4736.

Gross, C. H. and Shuman, S. (1996) “Vaccinia Virus RNA Helicase: Nucleic Acid Specificity in Duplex Unwinding, J. Virol, 70, 2615-2619.

Jankowsky, E. and Schwenzer, B. (1996) “Efficient Improvement of Hammerhead Ribozyme Mediated Cleavage of Long Substrates by Oligonucleotide Facilitators” Biochemistry 37, 15313-15321.

Jeltsch, A. and Pingoud, A. (1998) “Kinetic Characterization of Linear Diffusion of the Restriction Endonuclease EcoRV on DNA, Biochemistry 24, 2160-2169.

Kadare, G. and Haenni, A. L., (1997) “Virus encoded RNA Helicases,” J. Virol 71, 2583-2590.

Kelemen, B. R. and Raines, R. T. (1999) “Extending the Limits to Enzymatic Catalysis: Diffusion of Ribonuclease A in One Dimension” Biochemistry 27, 5302-5307.

Lohmann, T. M. and Bjoernson, K. P. (1996) “Mechanism of Helicase Catalyzed DNA Unwinding”, Annu. Rev. Biochem. 65, 169-214.

Moore, M. J. and Sharp, P. A. (1992) “Site Specific Modification of Pre-Messenger RNA-the 2′-Hydroxyl Groups at the Splice Sites”, Science, 256, 992-997.

Pyle, A. M., et al., (2000) “Using DNAzymes to Cut, Process and Map RNA Molecules For Structural Studies or Modification” Methods Enzymol 317, 140-146.

Santoro, S. W. and Joyce, G. F. (1997) “a General Purpose RNA-Cleaving DNA Enzyme”, Proc. Natl. Acad. Sci., USA, 94, 4262-4266.

Schnapp, B. Molecular Motors, (1995) “Two Heads Are Better Than one” Nature 373, 655-666.

Shimamoto, N. (1999) “One-Dimensional Diffusion of Proteins Along DNA, J. Biol. Chem. 274, 15239-15296.

Shuman, S. (1992) “Vaccinia Virus RNA Helicase: An Essential Enzyme Related to the DE-H Family of RNA Dependent NTPases”, Proc. Natl. Acad. Sci. USA, 89, 10935-10939.

Shuman, S. (1993) “Vaccinia Virus RNA Helicase, J. Biol. Chem., 268, 11798-11802.

Staley, J. P. and Guthrie, C. (1998) “Mechanical Devices of the Spliceosome: Motors Clocks Springs and Things” Cell 90, 1041-1050.

Wagner, I. D. O., et al., (1998) “The DEAH-Box Protein Prp 22 is an ATPase that Mediates ATP Dependant mRNA Release From the Spliceosome and Unwinds RNA Duplexes,” EMBO J. 17, 2926-2937.

Wincott et al. (1995) “Synthesis, Deprotection, Analysis and Purification of RNA and Ribozymes”, Nucleic Acids Res., 23, 2677-2684.

Xiang, Q., et al., (1998) “Sequence Specificity of a Group II Intron Ribozyme: Multiple Mechanisms For Promoting Unusually High Discrimination Against Mismatched Targets” Biochemistry 37, 3839-3849.

Zimmeric, C. and Frieden, C. (1998) “Analysis of Progress Curves by Simulations Generated by Numerical Integration” Biochem. I. 258, 381-387.

What is claimed is:

1. A method for detecting the release of a single-stranded RNA from an RNA duplex which comprises:
a) admixing an RNA helicase with the RNA duplex under conditions permitting the RNA helicase to unwind the RNA duplex and release single-stranded RNA, wherein the RNA duplex comprises a first RNA having a first label attached thereto and a second RNA, wherein said first label produces a luminescent energy pattern when the first RNA is present in the RNA duplex, which luminescent energy pattern differs from a luminescent energy pattern produced when the first RNA is not present in the RNA duplex; and
b) detecting a change in the luminescent energy pattern produced by the first label so as to thereby detect release of single-stranded RNA from the RNA duplex.

2. The method of claim 1, wherein in step (a) the conditions which permit the RNA helicase to unwind the RNA duplex and release single-stranded RNA comprise the presence of ATP and a divalent cation.

3. The method of claim 1, wherein the first label is present at the 5′ end of the first RNA.

4. The method of claim 1 or 3, wherein a second label is attached to the 3′ end of the second RNA and the luminescent energy pattern results from the interaction of luminescent energy released from the first label with the second label.

5. The method of claim 4, wherein the first and second label comprise fluorophors and the second label absorbs luminescent energy released from the first fluorophor.

6. The method of claim 5, wherein the first label is fluorescein iscothiocyanate and the second label is rhodamine isothiocyanate.

7. A method of measuring the rate of release of a single-stranded RNA from an RNA duplex which comprises detecting whether the single-stranded RNA is released from the RNA duplex at predetermined time intervals according to the method of claim 1, and determining therefrom the rate of release of the single-stranded RNA from the RNA duplex.

8. A method of determining whether a compound is capable of modulating the release of a single-stranded RNA from an RNA duplex by an RNA helicase which comprises detecting the release of the single-stranded RNA from the RNA duplex according to the method of claim 1, wherein the compound is added to the mixture of step (a).
